(19) United States
(12) Patent Application Publication
(10) Pub. No.: US 2007/0141687 A1
(43) Pub. Date: Jun. 21, 2007

(54) Increase in stress tolerance with ascorbic acid during fermentation

(76) Inventors: Danilo Porro, Erba (Como) (IT); Paola Branduardi, Milano (IT); Diethard Mattanovich, Wien (AT); Michael Sauer, Wien (AT)

Correspondence Address: RATNERPRESTIA, P.O. BOX 1596, WILMINGTON, DE, 19899, US

(21) Appl. No.: 11/546,951

(22) Filed: Oct. 12, 2006

Related U.S. Application Data
(63) Continuation-in-part of application No. 11/105,162, filed on Apr. 13, 2005.

(30) Foreign Application Priority Data
Apr. 7, 2006 (US) PCT/US06/12854

(51) Int. Cl. C12P 7/56; C12N 1/18; C12N 1/21; C12N 15/74

(52) U.S. Cl. 435/139; 435/252.3; 435/254.2; 435/483

(57) ABSTRACT

A method of increasing stress tolerance in recombinant organisms that have been engineered for industrial production is described. Stress tolerance is increased by making L-ascorbic acid available to the recombinant organism, either by exogenous addition to the culture medium or by endogenous production from D-glucose by the recombinant organism. To enable endogenous production, the recombinant organism is transformed with a coding region encoding a mannose epimerase (ME), a coding region encoding an L-galactose dehydrogenase (LGDH), and a D-arabinono-1,4-lactone oxidase (ALO). The recombinant organism may be further transformed with a myoinositol phosphatase (MIP).

### RELATED APPLICATIONS

This application is a continuation-in-part of U.S. Ser. No. 11/105,162, filed on Apr. 13, 2005, which is incorporated herein by reference. This application claims priority from U.S. Ser. No. 11/105,162 and from PCT/US06/012854, filed on Apr. 7, 2006, also incorporated herein by reference.

### FIELD OF THE INVENTION

The present invention relates generally to the field of increasing stress tolerance in organisms used for industrial production. More particularly, it relates to a process for making L-ascorbic acid available to organisms during industrial production.

### BACKGROUND

Microorganisms and cells can be easily grown on an industrial scale and are frequently employed in the commercial production of compounds such as organic acids, amino acids, vitamins, polyols, solvents, biofuels, therapeutics, vaccines, proteins, and peptides. Both prokaryotic and eukaryotic microorganisms are today easily and successfully used for the production of heterologous proteins as well as for the production of natural or engineered metabolites. Among prokaryotes, Escherichia coli and Bacillus subtilis are often used. Among eukaryotes, the yeasts, Saccharomyces cerevisiae and Kluyveromyces lactis, are often used. However, in an industrial process, wherein the organism is used as a means for production, stress on the organism typically leads to lower or zero production of the product, lower or zero productivity, a lower or zero yield of the product, or two or more thereof. Bacteria, yeast, other fungi, cultured animal cells, and cultured plant cells show similar responses to stress. (Close, D. C., et al., Oxidative Stress, Exercise, and Aging, H. M. Alessio, A. E. Hagerman, Eds. (2006), pp. 9-23; Sugiyama, K., et al., (2000), J Biol. Chem. 275, 15535-15540; Mongkolsuk, S. and Helmann, J. D. (2002), Molecular Microbiology 45, 9-15). Techniques for minimizing stress would therefore be useful for improving industrial production by these organisms.

Stresses may have cellular (internal or intracellular) origins, environmental (external or extracellular) origins, or both. Classical examples of the internally-originating stresses include protein and metabolite overproduction (in terms of weight/volume) and protein and metabolite overproductivity (in terms of weight/volume per unit time), among others. Examples of externally-originating stresses include high osmolarity, high salinity, oxidative stress, high or low temperature, non-optimal pH, presence of organic acids, presence of toxic compounds, and macro- and micro-nutrient starvation.

Stress is typically caused by stressors (or stimuli). Stressors are negative influences on a cell that require the cell to dedicate more effort to maintain equilibrium than is required in the absence of the stressor. This greater effort can lead to a higher or lower metabolic activity, lower growth rate, lower viability, or lower productivity, among other effects. Stressors are agents of a physical, chemical or biological nature that represent a change in the usual intracellular or extracellular conditions for any given life form. It follows that while a specific condition (e.g., a temperature of 65° C.) may be stressful (or even lethal) to a certain species that normally lives at 37° C., it may be optimal for a thermophilic organism.

At the cellular level, stress can damage DNA, lipids, proteins, membranes, and other molecules and macromolecules, induce apoptosis (programmed cell death), cell necrosis and cell lysis, and impair cell integrity and cell viability. These effects are often mediated by the generation of reactive oxygen species (ROS).

ROS can be generated through both intracellular and extracellular stimuli. The majority of endogenous ROS are produced through leakage of these species from the mitochondrial electron transport chain. In addition, cytosolic enzyme systems, including NADPH oxidases and by-products of peroxisomal metabolism, are also endogenous sources of ROS. Generation of ROS also can occur through exposure to numerous exogenous agents and events including ionizing radiation, UV light, chemotherapeutic drugs, environmental toxins, and hyperthermia. Oxidative damage caused by intracellular ROS can result in DNA base modifications, single- and double-strand DNA breaks, and the formation of apurinic/apyrimidinic lesions, many of which are toxic and/or mutagenic. Therefore, the resulting DNA damage may also be a direct contributor to deleterious biological consequences (Tiffany, B. et al., (2004) Nucleic Acids Research 32, 3712-3723).

One example of an industrial process known to be hampered by stress responses is the production of lactic acid by bacteria or yeast. During a typical lactic acid fermentation, the accumulation of lactic acid in the medium also causes a drop in pH of the medium. The stress of low pH is amplified by the ability of the organic free acid to diffuse through the membrane and dissociate in the higher pH of the cytoplasm. The accumulation of lactic acid inhibits cell growth and metabolic activity. The toxicity of these stresses is mediated at least in part by reactive oxygen species. As a result, the extent of lactic acid production is greatly reduced by the accumulation of lactic acid in the medium.

The addition of Ca(OH)₂, CaCO₃, NaOH, or NH₄OH to the fermentation medium to neutralize the lactic acid and to thereby prevent the pH drop is a conventional operation in industrial processes to counteract the negative effects of free lactic acid accumulation. These processes allow the production of lactate(s) by maintaining the pH at a constant value in the range of about 5 to 7, which is well above the pKa of lactic acid (3.86).

However, this neutralization procedure has major disadvantages. Additional operations are required to regenerate free lactic acid from its salt and to dispose of or recycle the neutralizing cation, which adds expense to the process. The added operations and expense could be lessened if free lactic acid could be accumulated by organisms growing at low pH values. To this end, the use of recombinant yeast that are engineered for industrial production of free lactic acid, and, in particular, recombinant yeast from strains showing greater tolerance for extreme environmental conditions have been described. Engineered strains of recombinant yeast functionally transformed with a gene for lactate dehydrogenase (LDH) in the genera Saccharomyes, Zygosaccharomyces, Torulaspora, and Kluveromyces have been produced as described in U.S. Pat. Nos. 6,429,006 and 7,049,108. While these recombinant strains show improved efficiency of lactic acid production at low pH, they are still adversely affected by stresses. In addition, it may be necessary to use organisms or strains that are less tolerant of extreme environmental conditions for the industrial production of specific compounds.

Ascorbic acid is a known antioxidant that is produced in all higher plants and many higher animals. Ascorbic acid has been shown to modulate the heat shock response in yeast through an effect on ROS(C. Moraitis and B. P. G. Curran. (2004), Yeast 21, 313-323), and to improve cell viability and reduce proteolysis of the end product of high cell-density fermentation (Xiao, A. et al. (2006), Appl. Microbiol. Biotechnol. 72, 837-844). These effects suggest that ascorbic acid could improve stress tolerance in general in organisms utilized for industrial production.

We have shown that recombinant yeast that are functionally transformed to produce L-ascorbic acid, the biologically active enantiomer, from D-glucose produce lower levels of ROS and exhibit improved growth and viability under conditions of low pH, oxidative stress, and in the presence of high concentrations of lactic acid. (Branduardi, P., et al., International Specialised Symposium on Yeast. ISSY25, Systems Biology of Yeast—From Models to Applications. “L-ascorbic acid production from D-glucose in metaboloic engineered Saccharomyces cerevisiae and its effect on strain robustness.” Hanasaari, Espoo, Finland, Jun. 21, 2006).

Accordingly, it would be advantageous to industrial fermentation processes if the organisms and cells used for industrial production could endogenously produce L -ascorbic acid from D-glucose.

### SUMMARY OF THE INVENTION

The present invention relates to a method of increasing stress tolerance in a recombinant organism that is engineered for industrial production of at least one product. The method comprises making L-ascorbic acid available to the recombinant organism.

In one embodiment, ascorbic acid is made available by functionally transforming the recombinant organism with a coding region encoding a mannose epimerase (ME), a coding region encoding an L-galactose dehydrogenase (LGDH), and a coding region encoding a D-arabinono-1,4-lactone oxidase (ALO). In a further embodiment, the functionally transformed, recombinant organism is further functionally transformed with a coding region encoding a myoinositol phosphatase (MIP).

In another embodiment, the L-ascorbic acid is made available by culturing the recombinant organism in culture medium containing an effective amount of L-ascorbic acid.

### DESCRIPTION OF THE DRAWINGS

FIG. 1 shows the main plant pathway for the synthesis of L-ascorbic acid from D-glucose.

FIG. 2 shows the optical density at 660 nm of BY4742 (▴) and YML007w (yap1 mutant strain) (∘) yeast in the absence (FIG. 2a) and presence (FIGS. 2b-2c) of oxidative stress. Yap1 activates genes required for the response to oxidative stress; deletion of this gene leads to the observed phenotype.

FIG. 3 shows the impact of two stressors on yeast growth. FIGS. 3a-3b show the optical density at 660 nm of BY4742 wt (▴) and YML007w (∘) yeast in the presence of H₂O₂ in medium +/−ascorbic acid. FIG. 3c shows the optical density at 660 nm of wild type yeast GRFc, CEN.PK 113-5D, and BY4741 in the presence of 40 g/l lactic acid and zero, or increasing levels of ascorbic acid.

FIG. 4 shows the optical density at 660 nm of BY4742 wt (▴); YML007w expressing ALO, LDGH and ME (□); and YML007w expressing ALO, LDGH, ME and MIP (▪) yeasts in the presence of oxidative stress (FIGS. 4a-4b).

FIG. 5 shows the optical density at 660 nm of wild type GRFc (▴); GRF18U expressing ALO, LDGH and ME (□); and GRF18U expressing ALO, LDGH, ME and MIP (▪) yeast strains in the absence (FIG. 5a) and presence (2 mM of H₂O₂) of oxidative stress. (FIG. 5b).

FIG. 6 shows ROS (upper panels) and viability (bottom panels) determination by flow cytometric analyses of S. cerevisiae cells producing (YML007w ALO, LDGH, ME, MIP, open area) or not producing (YML007w, full area) ascorbic acid when grown in minimal glucose medium in the presence (right) or absence (left) of hydrogen peroxide.

FIG. 7 shows growth curves of strains BY4742c (□) and BY4742 ALO, LDGH, ME, MIP (▪) inoculated in minimal glucose medium at pH 2.2 (a), or in minimal glucose medium pH 3.0 containing 38 g/l of lactic acid (b).

FIG. 8 shows growth curves of strains BY4742c (□) and BY4742 ALO, LDGH, ME, MIP (▪) that were first grown for 24 h in minimal glucose medium under nonlimiting conditions, and then transferred to minimal glucose medium at pH 2.2 (a), or to minimal glucose medium pH 3 containing 38 g/l of lactic acid (b).

FIG. 9 shows growth curves, as measured by OD660, and lactic acid production by S. cerevisiae strain NRRL Y-30696 grown in minimal glucose medium containing 2.78 g/L CaCO₃ and increasing concentrations of ascorbic acid (AA). 0 g/L AA (□), 0.16 g/L AA (+), 0.3 g/L AA (▴), or 0.6 g/L (♦)

### DETAILED DESCRIPTION

The present invention relates to a method of increasing stress tolerance in recombinant cells or organisms that have been engineered for the industrial production of products such as organic acids, amino acids, vitamins, polyols, solvents, biofuels, therapeutics, vaccines, proteins, and peptides by increasing the available amount of ascorbic acid.

A “recombinant” cell or organism is one that contains a nucleic acid sequence that is not naturally occurring in that cell or organism, or one that contains an additional copy or copies of an endogenous nucleic acid sequence, wherein the nucleic acid sequence is introduced into the cell or organism or into an ancestor cell thereof by human action. Introduction of the gene into the cell or organism is known as “transformation” and the recipient organism or cell is said to be “transformed.” Recombinant DNA techniques are well-known to those of ordinary skill in the art, who will also understand how to choose appropriate vectors and promoters for the transformation of particular organisms or strains. (For example, see methods in Sambrook, J. and Russell, D. W., Molecular Cloning: A Laboratory Manual, 3^{rd} Edition, Cold Spring Harbor Laboratory Press, 2001). Very basically, a coding region of the homologous and/or heterologous gene is isolated from a “donor” organism that possesses the gene. The recombinant organism, as well as the donor, may be a prokaryote, such as a bacterium, or a eukaryote, such as a protozoan, alga, fungus, plant, or animal.

In one well-known technique, a coding region is isolated by first preparing a genomic DNA library or a cDNA library, and second, identifying the coding region in the genomic DNA library or cDNA library, such as by probing the library with a labeled nucleotide probe that is at least partially homologous with the coding region, determining whether expression of the coding region imparts a detectable phenotype to a library microorganism comprising the coding region, or amplifying the desired sequence by PCR. Other techniques for isolating the coding region may also be used.

Methods for preparing recombinant nucleotides and transferring them into a host organism are well-known to those of ordinary skill in the art. Briefly, the desired coding region is incorporated into the recipient organism in such a manner that the encoded protein is produced by the organism in functional form. That is, the coding region is inserted into an appropriate vector and operably linked to an appropriate promoter on the vector. If necessary, codons in the coding region may be altered, for example, to create compatibility with codon usage in the target organism, to change coding sequences that can impair transcription or translation of the coding region or stability of the transcripts, or to add or remove sequences encoding signal peptides that direct the generated protein to a specific location in or outside the cell, e.g., for secretion of the protein. Any type of vector, e.g., integrative, chromosomal, or episomal, may be used. The vector may be a plasmid, cosmid, yeast artificial chromosome, virus, or any other vector appropriate for the target organism. The vector may comprise other genetic elements, such as an origin of replication to allow the vector to be passed on to progeny cells of the host carrying the vector, sequences that facilitate integration into the host genome, restriction endonuclease sites, etc. Any promoter active in the selected organism, e.g., homologous, heterologous, constitutive, inducible, or repressible may be used. An “appropriate” vector or promoter is one that is compatible with the selected organism and will generate a functional protein in that organism. The recombinant organism thus transformed is referred to herein as being “functionally transformed.”

The recombinant cells and organisms of the invention can be obtained by any method allowing a foreign DNA to be introduced into a cell, for example, transformation, electroporation, conjugation, fusion of protoplasts or any other known technique (Spencer J. F. et al. (1988), Journal of Basic Microbiology 28, 321-333). A number of protocols are known for transforming yeast, bacteria, and eukaryotic cells. Transformation can be carried out by treating the whole cells in the presence of lithium acetate and of polyethylene glycol according to Ito H. et al. ((1983), J. Bacteriol., 153:163), or in the presence of ethylene glycol and dimethyl sulphoxyde according to Durrens P. et al. ((1990) Curr. Genet., 18:7). An alternative protocol has also been described in EP 361991. Electroporation can be carried out according to Becker D. M. and Guarente L. ((1991) Methods in Enzymology, 194:18). The use of non-bacterial integrative vectors may be preferred when the yeast biomass is used at the end of the fermentation process as stock fodder or for other breeding, agricultural or alimentary purposes.

The transformed organism is propagated in an appropriate culture medium. Culturing techniques and specialized media are well known in the art. For industrial production, the organism is preferably cultured in an appropriate medium in a fermentation vessel.

Organisms frequently utilized for industrial production are yeast and bacteria. Yeast to be transformed can be selected from any known genus and species of yeast. Yeast species are described by N. J. W. Kreger-van Rij, (“The Yeasts,” (1987) Biology of Yeasts, A. H. Rose and J. S. Harrison, Eds. London: Academic Press, Chapter 2) In one embodiment, the yeast genus is selected from the group consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces. In another embodiment, the yeast is selected from S. cerevisiae strains, including GRF18U, W3031B, BY4742 (MATα; his3; leu2, lys2; ura3, EuroScarf Accession No. Y10000); Z. bailii ATCC 60483; K. lactis PM6-7A; BY4741 (MATα; his3; leu2; met15; ura3, Euroscarf Accession No. Y00000), CEN.PK 113-5D (MATα ura3-52; cir+), and yeast strains engineered to produce lactic acid, including NRRL Y-30696, NRRL Y-30698, NRRL Y-30742; K. lactis PM6-7/pEPL2, PMI/C1[pELP2]; Zygosaccharomyces bailii ATTC36947/pLAT-ADH, ATCC60483/pLAT-ADH.

Yeast have been widely utilized in the production of products. Yeast biomass is an important product as cultures for development of food products as well as a nutrient rich food and feed component. Genetic engineering has broadened the value of yeast production systems providing a route to organic acids (Porro, D. et al. (2002), U.S. Pat. No. 6,429,006); vitamins (Shiuan, D., US2003/0104584); polyols (Geertman, J. M, et al., (2006) Metabolic Engineering, June 30:(Epublication); biofuel (Ho, N. W. Y. and Tsao, G. T. (1998), U.S. Pat. No. 5,789,210); (Bosman, F., et al. (2006) U.S. Pat. No. 7,048,930); proteins (Gerard, G. F., et al. (2006). U.S. Pat. No. 7,115,406); and peptides (Lee, S. Y., et al., Lett. Appl. Microbiol (2003), 36, 121-128.).

Bacteria to be transformed can be selected from any known genus and species of the Eubacteria or the Archaea (also encompassed herein by the term, “bacteria”). Bacteria are cataloged at the NCBI Taxonomy website: http://www.ncbi.nlm.nih.gov/entrez/query.fcgi?db=Taxonomy. In one embodiment the bacteria can be selected from the genera Bacillus, Escherichia, Lactobacillus, Lactococcus, Pseudomonas, or Acetobacter.

Bacteria have been widely utilized to produce industrial products. The natural range of available products has been extended by mutagenesis and screening and further by genetic engineering. Bacteria provide routes to organic acids (WO2006/083410); amino acids (WO2005/090589); vitamins (Santos, et al., Abstracts of Papers, 232nd ACS National Meeting, San Francisco, Calif., United States, Sep. 10-14, 2006, BIOT-243); polyols (Dunn-Coleman, N. S., et al. (2006) U.S. Pat. No. 7,074,608); solvents (Harris, L. M., et al. (2001), Journal of Industrial Microbiology & Biotechnology 27, 322-328); biofuels (Ingram, L. O. and Zhou, S. WO2000/071729); therapeutics (Pizza, M., et al. (2006) U.S. Pat. No. 7,115,730); proteins (Gerard, G. F., et al. (2006) U.S. Pat. No. 7,115,406); and peptides (Knapp, S., et al. (1992) U.S. Pat. No. 5,159,062).

Filamentous fungi are widely utilized to produce organic acids (Bizukojc, M. and Ledakowicz, S., Process Biochemistry (2004), 39, 2261-2268.); and proteins (Wang, L., et al., (2003) Biotechnology Advances 23, 115-129). Filamentous fungi to be transformed can be selected from any known genus and species. Fungi are cataloged at the NCBI Taxonomy Website: http://www.ncbi.nlm.nih.gov/Taxonomy/Browser/wwwtax.cgi?id=4751.

In one embodiment the filamentous fungi can be selected from the genera Rhizopus, Aspergillus, or Trichoderma.

In one embodiment of the invention, the recombinant organism is functionally transformed with coding regions that encode a mannose epimerase (D-mannose:L-galactose epimerase; ME), L-galactose dehydrogenase (LGDH); and D-arabinono-1,4-lactone oxidase (ALO). These coding sequences enable the recombinant organism to produce enzymes necessary for the endogenous production of L-ascorbic acid from D-glucose. As a result of transformation with ME, LGDH, and ALO, and endogenous production of L-ascorbic acid, the organism shows increased tolerance to stress when compared with a strain of the same organism that cannot produce L-ascorbic acid.

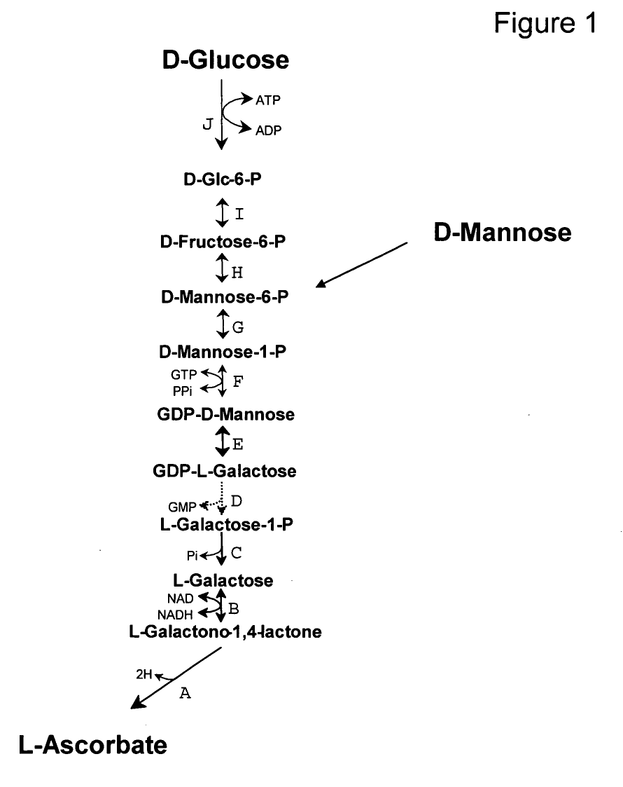

An ME is any GDP-mannose-3,5-epimerase (5.1.3.18), that is any enzyme that catalyzes the conversion of GDP-mannose to GDP-L-galactose (FIG. 1). An exemplary ME is encoded by the sequence listed as SEQ ID NO:1.

In one embodiment, the ME has at least about 95% identity with SEQ ID NO:1. “Identity” can be determined by a sequence alignment performed using the ClustalW program and its default values, namely: DNA Gap Open Penalty=15.0, DNA Gap Extension Penalty=6.66, DNA Matrix=Identity, Protein Gap Open Penalty=10.0, Protein Gap Extension Penalty=0.2, Protein matrix=Gonnet. Identity can be calculated according to the procedure described by the ClustalW documentation: “A pairwise score is calculated for every pair of sequences that are to be aligned. These scores are presented in a table in the results. Pairwise scores are calculated as the number of identities in the best alignment divided by the number of residues compared (gap positions are excluded). Both of these scores are initially calculated as percent identity scores and are converted to distances by dividing by 100 and subtracting from 1.0 to give number of differences per site. We do not correct for multiple substitutions in these initial distances. As the pairwise score is calculated independently of the matrix and gaps chosen, it will always be the same value for a particular pair of sequences.”

In another embodiment, the recombinant organism transformed with the coding sequences for ME, LGDH, and ALO is further functionally transformed with a coding region encoding a myoinositol phosphatase (MIP). An MIP is any myoinositol phosphatase (3.1.3.25), that also catalyzes the conversion of L-galactose-1P to L-galactose. L-galactose-1-phosphatase has been annotated as inositol/myo-inositol monophosphatase galactose-1-phosphatase and may be referred to as MIP/VTC4 (Conklin, P. L. et al. (2006) J. Biol. Chem. 281, 15662-70). In one embodiment, the MIP has at least about 95% identity with SEQ ID NO:2. Identity is determined as described above.

In another embodiment, the recombinant organism is further transformed with a coding region encoding an enzyme selected from L-galactono-1,4-lactone dehydrogenase (AGD), D-arabinose dehydrogenase (ARA) or L-gulono-1,4-lactone oxidase (GLO), as described, for example, in U.S. Pat. No. 6,630,330, which is incorporated herein by reference.

Although the pathway for the production of ascorbic acid in plants is shown in FIG. 1, the present invention is not limited to the enzymes of the pathways known for the production of L-ascorbic acid intermediates or L-ascorbic acid in plants, yeast, or other organisms. (Examples of known L-ascorbic acid pathways in plants and animals are described in Conklin, P. L., et al. (2006), J. Biol. Chem. 281, 15662-15670; and in Valpuesta, V. and Botella, M. A. (2004) Trends in Plant Science 12, 573-577). One of ordinary skill in the art will understand that increasing flux through any pathway resulting in L-ascorbic acid biosynthesis will result in production of higher levels of L-ascorbic acid. This can be accomplished by increasing the levels of enzymes in the pathway that are limiting.

The coding regions for any of the desired enzymes may be isolated from any source or may be chemically synthesized. Following transformation with the coding regions for ME, LGDH, and ALO, (with or without the coding region for MIP), the recombinant organism is cultured in medium containing a carbon source that can be converted to L-ascorbic acid, such as D-glucose.

When the recombinant organism for industrial production is a eukaryotic organism, it is important to ensure that each of the enzymes used to produce ascorbic acid is appropriately compartmentalized in the eukaryotic cell. This is accomplished by including sequences encoding targeting labels in the recombinant vector. These types of sequences are disclosed, for example, in Alberts, B., et al., Molecular Biology of the Cell, 4^{th} Edition, New York: Garland Science Publ., 2002, pages 659-710.

With respect to the invention, “production” means the process of making one or more products using a recombinant organism. Production can be quantified at any moment in time after commencement of the process by determining the weight of a product produced per weight or volume of the medium on which the recombinant organism's growth and survival is maintained, or weight or volume of the recombinant organism's biomass. “Productivity” means the amount of production, as quantified above, over a given period of time (e.g., a rate such as g/L per hour, mg/L per week, or g/g of biomass per hour). “Yield” is the amount of product produced per the amount of substrate converted into the product. This definition of “yield” also applies to endogenous production of L-ascorbic acid.

Stress tolerance, as used herein, may manifest as a decrease in the negative impact of stress on the organism, such as a decline in the production of ROS or a positive effect on productivity, yield, or production. An increase in stress tolerance can be measured by a number of parameters, for example, as an increase in growth rate, an increase in cell density, a decrease in the inhibition of productivity, an increase in viability, an increase in metabolism, or an increase in yield, productivity, or production. An “effective amount” of L-ascorbic acid is an amount of L-ascorbic acid present in the culture medium that gives rise to an improvement in stress tolerance as measured by any of these parameters, when compared with stress tolerance of the organism grown in medium that does not contain L-ascorbic acid.

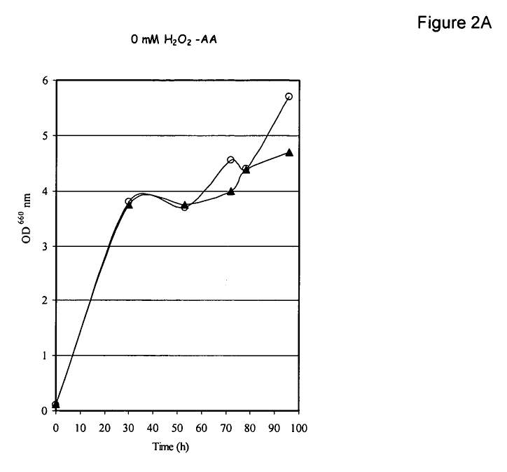

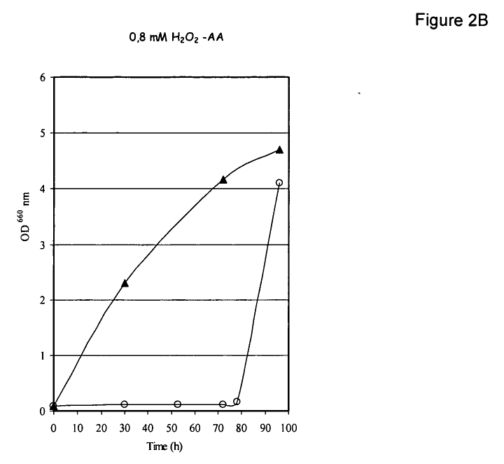

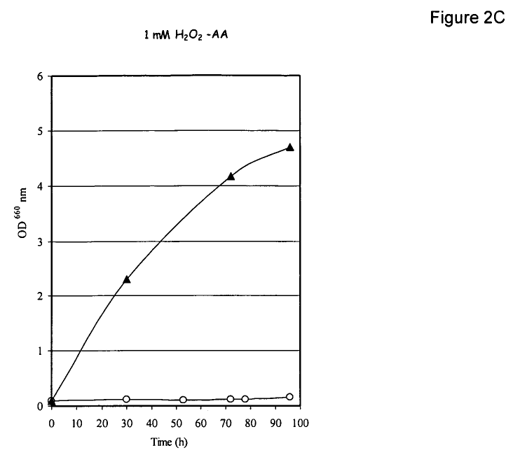

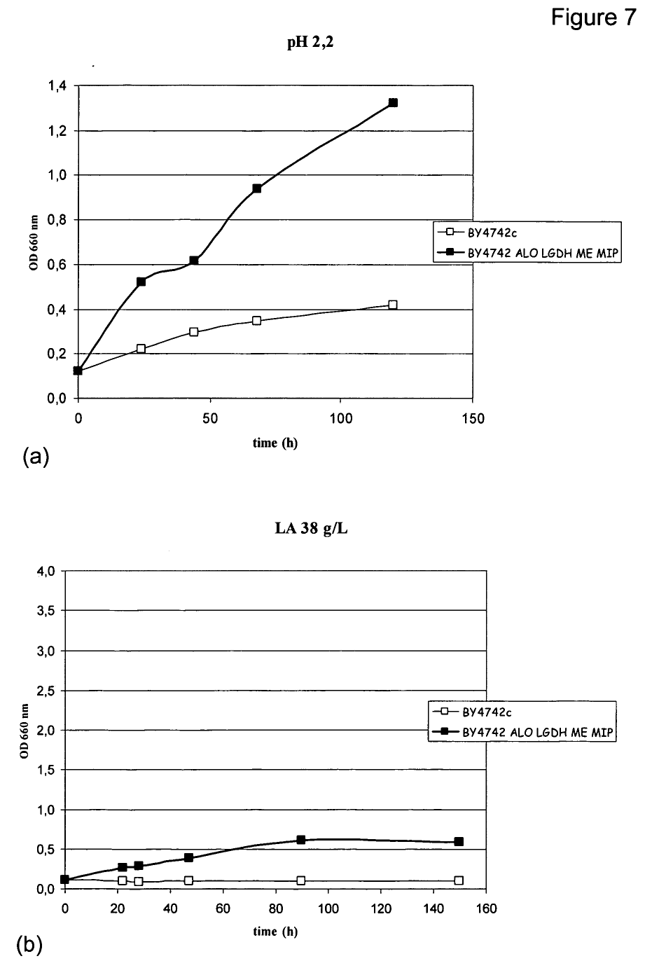

As shown in FIGS. 2-5, yeast transformed with coding sequences for ME, LGDH, and ALO, or with this group of coding sequences plus a coding sequence for MIP, have greater stress tolerance than yeast that are not so transformed. FIG. 7 shows that endogenously produced L-ascorbic acid correlates with increased tolerance to low pH and oxidative stresses. This increased stress resistance can manifest as one or more of increased growth rate of the transformed organism, increased viability of the transformed organism, or increased production by the transformed organism.

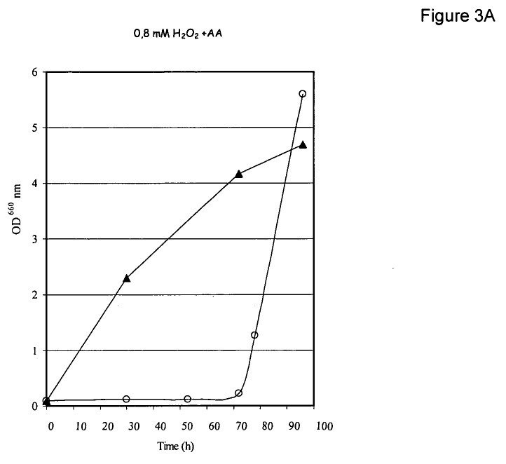

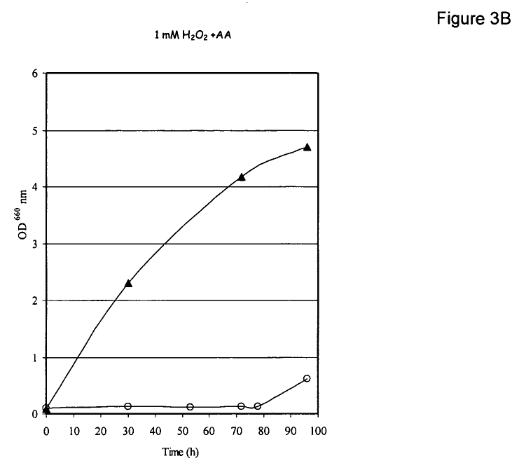

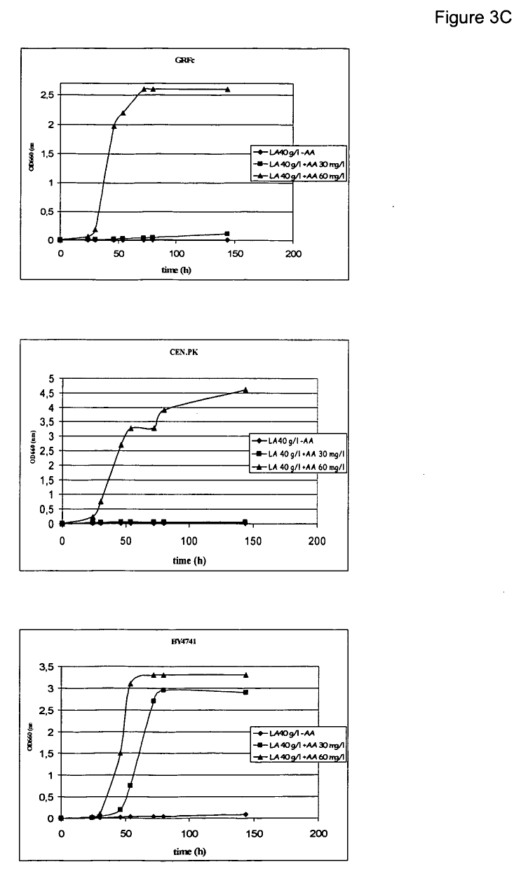

We also show, in FIG. 3, that the addition of L-ascorbic acid to the fermentation medium improves stress tolerance, in particular, tolerance to low pH and oxidative stress. Accordingly, in one embodiment of the invention, the available amount of ascorbic acid is increased by adding L-ascorbic acid to the fermentation medium. Exogenous L-ascorbic acid may be added to cultures that do or do not produce L-ascorbic acid endogenously.

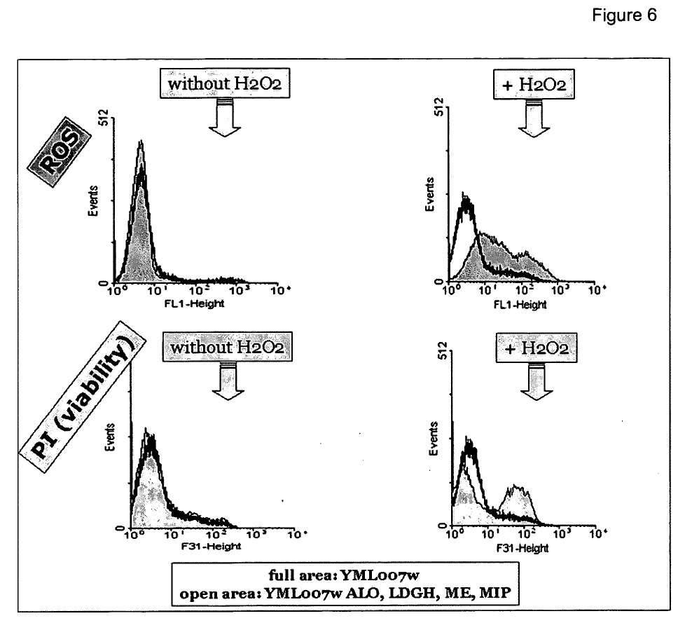

Though not wishing to be bound by a single theory, we suggest that the increased stress tolerance results from an increase in antioxidant levels (specifically, L-ascorbic acid) and a reduction in the levels of endogenous reactive oxygen species (ROS) in the organism, imparting greater resistance to oxidative stress, as shown in FIG. 6. The increased stress tolerance makes organisms that endogenously produce ascorbic acid particularly suitable for industrial production. Such organisms include plant and animal cells that produce ascorbic acid either naturally or through genetic engineering. (e.g., organisms described in Valpuesta, V. and Botella, M. A. (2004) Trends in Plant science 9, 573-577 and genetically engineered plant and animal cells.)

Organisms with increased stress tolerance that are to be used for industrial production may be created by any methods known to those of skill in the art for engineering recombinant organisms. The organism may be co-transformed with the necessary coding regions for production of L-ascorbic acid (i.e., ME, LGDH, ALO+/−MIP) and the coding sequences for the industrial product that the organism will produce. The organism may first be engineered to express the L-ascorbic acid coding sequences and then subsequently be transformed with coding regions for the industrial product. Alternatively, the organism may first be engineered to produce the industrial product and subsequently be transformed with the coding regions for production of L-ascorbic acid.

Endogenous production of L-ascorbic acid by the recombinant organism is particularly useful if the recombinant organism is cultivated under conditions of osmotic, pH, temperature, or oxidative stress. Osmotic stress is a condition in which the organism or cell encounters a difference in osmolarity from the optimal osmolarity defined for the respective microorganism. For example, in the yeast S. cerevisiae, an osmolarity greater than 500 mOsmol leads to a stress response.

A pH stress occurs if an organism or strain of organism encounters a difference in pH value from the optimal pH value for that strain of more than one to three pH units. For example, in the wild type strain of the yeast S. cerevisiae, the typical optimal pH for performance of bioprocesses is 5.0. A pH of less than 4.0 or more than 6.0 may cause a stress response in this strain that can affect the transcription of pH sensitive genes.

Temperature stress is a condition in which the organism encounters a cultivation temperature different the optimal temperature value for growth or production for a particular organism. In the yeast, S. cerevisiae, a temperature at or above 32° C. can cause stress responses. For the bacterium E. coli, a temperature at or above 38° C. can lead to stress responses.

Oxidative stress is a general term used to describe the steady state level of oxidative damage in a cell, caused by the reactive oxygen species (ROS). This damage can affect a specific molecule or the entire organism. Reactive oxygen species, such as free radicals and peroxides, represent a class of molecules that are derived from the metabolism of oxygen and exist inherently in all aerobic organisms. Oxidative stress results from an imbalance between formation and neutralization of pro-oxidants. Animal cells, as well as single-celled organisms, can be exposed to significant oxidative stress during standard cell culture conditions.

Endogenous production of L-ascorbic acid is also particularly useful in a cell or organism if it is subjected to stress due to overproduction of a metabolite or a protein. Such stresses may be indicated, for example, by the upregulation of genes related to the UPR (unfolded protein response), which is known in the art. (Foti, D. M., et al. (1999) J. Biol. Chem. 274, 30402-30409).

In one embodiment, the recombinant organism may be a yeast that has been engineered to produce and secrete lactic acid. The applications of lactic acid and its derivatives encompass many fields of industrial activities (e.g., chemistry, cosmetics, and pharmacy), as well as important aspects of food manufacture and use. Furthermore, today there is growing interest in the production of such an organic acid to be used directly for the synthesis of biodegradable polymer materials.

Lactic acid may be produced by chemical synthesis or by fermentation of carbohydrates using single-celled organisms. The latter method is now commercially preferred because organisms have been developed that produce exclusively one isomer, as opposed to the racemic mixture generated by chemical synthesis. The most important non-recombinant industrial organisms currently used to produce lactic acid, such as species of the genera Lactobacillus, Bacillus, and Rhizopus, produce L(+)-lactic acid. Production by fermentation of D(−)-lactic acid or mixtures of L(+)- and D(−)-lactic acid are also known.

During a typical lactic acid fermentation, the accumulation of lactic acid in the medium is detrimental to metabolic activity. In addition, the accumulation of lactic acid lowers the pH of the medium, which also inhibits cell growth and metabolic activity. As a result, the extent of lactic acid production is reduced as the lactic acid product accumulates.

Methods for the construction of recombinant yeasts expressing at least one copy of a lactate dehydrogenase (LDH) gene, which shifts the glycolytic flux towards the production of lactic acid, have been described in U.S. Pat. Nos. 6,429,006 and 7,049,108, both of which are incorporated herein by reference. These references report that lactic acid can be produced by metabolically modified yeasts belonging to the genera of Kluyveromyces, Saccharomyces, Torulaspora and Zygosaccharomyces. While any yeast species could be used, these species are preferred because these strains can grow and/or metabolize at very low pH, especially in the range of pH 4.5 or less. In addition, genetic engineering methods for these strains are well-developed, and these strains are widely accepted for use in food-related applications.

The yield of lactic acid can be increased by increasing copy numbers of the LDH gene in each yeast. Higher yields (>80% g/g) of lactic acid may be obtained from these engineered yeast strains if both the ethanolic fermentation pathway and the use of pyruvate by mitochondria are replaced by lactic fermentation. The recombinant yeast can also be transformed to overexpress a lactate transporter, for example, the JEN1 gene encoding for the lactate transporter of S. cerevisiae, can to ensure secretion of the product.

The expression of a LDH gene in yeast strains allows the production of lactic acid at acid pH values so that the free acid is directly obtained and the cumbersome conversion and recovery of lactate salts are minimized. In this invention, the pH of the fermentation medium may initially be higher than 4.5, but will decrease to a pH of 4.5 or less, preferably to a pH of 3 or less at the termination of the fermentation.

The gene coding for LDH may be from any species (e.g., mammalian, such as bovine, or bacterial), and it may code for the L(+)-LDH or the D(−)-LDH. Alternatively, both types of LDH genes may be expressed simultaneously. In addition, any natural or synthetic variants of LDH DNA sequences, any DNA sequence with high identity to a wild-type LDH gene, any DNA sequence complementing the normal LDH activity may be used.

The co-expression of ascorbic acid in a lactic acid producing microorganism to improve the stress tolerance and robustness of that organism could be accomplished by introduction of ME, LGDH, ALO, and, optionally, MIP. The transformation of the yeast strains could be carried out by means of either integrative or replicative plasmid or linear vectors. In a particular embodiment of the invention, the recombinant DNA is part of an expression plasmid which can be of autonomous or integrative replication.

For the production of lactic acid, the recombinant yeast strains that endogenously produce ascorbic acid and produce and secrete lactic acid would be cultured in a medium containing a carbon source, D-glucose, and other essential nutrients. The lactic acid would be recovered at a pH of 7 or less, preferably at a pH of 4.5 or less, and even more preferably at a pH of 3 or less. Because the pH of the culture medium would be reduced, less neutralizing agent would be required. The formation of lactate salt would be correspondingly reduced and proportionally less regeneration of free acid would be necessary in order to recover lactic acid.

Because the recombinant yeast are more stress tolerant due to the endogenous production of L-ascorbic acid, the yeast cells separated from the lactic acid product could be utilized again as seed microorganisms for a fresh lactic acid fermentation. In addition, the yeast cells could be continuously separated and recovered during the lactic acid fermentation, and hence, the fermentation could be carried out continuously at low pH with less severe effects of pH and oxidative stress on yeast viability, production, productivity, and yield.

The following definitions are provided in order to aid those skilled in the art in understanding the detailed description of the present invention.

“Ascorbic acid” as well as “ascorbate” as used herein, refers to L-ascorbic acid.

“Ascorbic acid precursor” is a compound that can be converted by an organism of the present invention, either directly or through one or more intermediates, into L-ascorbic acid.

“Amplification” refers to increasing the number of copies of a desired nucleic acid molecule or to increase the activity of an enzyme, by whatsoever means.

“Codon” refers to a sequence of three nucleotides that specify a particular amino acid.

“DNA ligase” refers to an enzyme that covalently joins two pieces of double-stranded DNA.

“Electroporation” refers to a method of introducing foreign DNA into cells that uses a brief, high voltage DC charge to permeabilize the host cells, causing them to take up extra-chromosomal DNA.

“Endonuclease” refers to an enzyme that hydrolyzes double stranded DNA at internal locations.

“Engineered for industrial production” refers to a recombinant organism that has been genetically modified to produce an industrial product.

Enzyme 1.1.3.37, D-arabinono-1,4-lactone oxidase, refers to a protein that catalyzes the conversion of D-arabinono-1,4-lactone+O₂ to D-erythroascorbate+H₂O₂. The same enzyme due to broadness of substrate range catalyses the conversion of L-galactono-1,4-lactone+O₂ to L-ascorbic acid+H₂O₂. Erroneously the same enzyme is referred to as L-galactono-1,4-lactone oxidase (enzyme 1.1.3.24) (Huh, W. K. et al. (1998), Mol. Microbiol. 30, 895-903)

Enzyme 1.3.2.3, L-galactono-1,4-lactone dehydrogenase, refers to a protein that catalyzes the conversion of L-galactono-1,4-lactone+2 ferricytochrome C to L-ascorbic acid+2 ferrocytochrome C.

Enzyme 1.1.3.8, L-gulono-1,4-lactone oxidase, refers to a protein that catalyzes the oxidation of L-gulono-1,4-lactone to L-xylo-hexulonolactone which spontaneously isomerizes to L-ascorbic acid.

Enzyme GDP-mannose-3,5-epimerase (5.1.3.18), refers to a protein that catalyzes the conversion of GDP-mannose to GDP-L-galactose.

Enzyme myoinositol phosphatase (3.1.3.23), refers to a protein that catalyzes the conversion of L-galactose-1P to L-galactose. L-galactose-1-phosphatase has been annotated as inositol/myo-inositol monophosphatase galactose-1-phosphatase and may be referred to as MIP/VTC4 (Conklin, P. L. (2006) J. Biol. Chem. 281, 15662-70).

Other enzymes of interest, and their classification numbers, are as follows:

| | |
|---|---|
| GDP-Mannose 3,5-epimerase | 5.1.3.18 |
| L-Galactono-1,4-lactone dehydrogenase | 1.3.2.3 |
| UDP-Glucuronate 4-epimerase | 5.1.3.6 |
| L-Gulono-1,4-lactone oxidase | 1.1.3.8 |
| Myoinositol 1-P monophosphatase | 3.1.3.25 |
| UDP-Glucose 4-epimerase | 5.1.3.2 |
| D-arabinose 1-dehydrogenase (NAD) | 1.1.1.116 |
| D-arabinose 1-dehydrogenase (NADP) | 1.1.1.117 |

The term “expression” refers to the transcription of a gene to produce the corresponding mRNA and translation of this mRNA to produce the corresponding gene product, i.e., a peptide, polypeptide, or protein.

The term “fermentation” refers to a process in which organisms growing in a liquid or solid medium produce an industrial product. As used herein, the term does not refer exclusively to non-oxidative metabolism.

The phrase “functionally linked” or “operably linked” refers to a promoter or promoter region and a coding or structural sequence in such an orientation and distance that transcription of the coding or structural sequence may be directed by the promoter or promoter region.

The phrase “functionally transformed” refers to an organism that has been transformed with an exogenous nucleic acid and is capable of producing a functional protein or peptide encoded by that amino acid.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

The term “genome” encompasses both the chromosomes and plasmids within a host cell. Encoding DNAs of the present invention introduced into host cells can therefore be either chromosomally integrated or plasmid-localized.

“Heterologous DNA” refers to DNA from a source different than that of the recipient cell.

“Homologous DNA” refers to DNA from the same source as that of the recipient cell.

“Hybridization” refers to the ability of a strand of nucleic acid to join with a complementary strand via base pairing. Hybridization occurs when complementary sequences in the two nucleic acid strands bind to one another.

The term “medium” refers to the chemical environment of the organism, comprising any component required for the growth of the organism and one or more precursors for the production of ascorbic acid. Components for growth and precursors for the production of ascorbic acid may or may be not identical.

“Open reading frame (ORF)” refers to a region of DNA or RNA encoding a peptide, polypeptide, or protein.

“Plasmid” refers to an extra chromosomal, replicatable piece of DNA.

“Polymerase chain reaction (PCR)” refers to an enzymatic technique to create multiple copies of one sequence of nucleic acid. Copies of DNA sequence are prepared by shuttling a DNA polymerase between two amplimers. The basis of this amplification method is multiple cycles of temperature changes to denature, then re-anneal amplimers, followed by extension to synthesize new DNA strands in the region located between the flanking amplimers.

The term “promoter” or “promoter region” refers to a DNA sequence, usually found upstream (5′) to a coding sequence, that controls expression of the coding sequence by controlling production of messenger RNA (mRNA) or other functional RNAs, (e.g., tRNAs, rRNAs, sRNAs), by providing the recognition site for RNA polymerase and/or other factors necessary for start of transcription at the correct site.

A “recombinant cell” or “transformed cell” is a cell that contains a nucleic acid sequence not naturally occurring in the cell or an additional copy or copies of an endogenous nucleic acid sequence, wherein the nucleic acid sequence is introduced into the cell or an ancestor thereof by human action.

The term “recombinant vector” or “recombinant DNA or RNA construct” refers to any agent such as a plasmid, cosmid, virus, autonomously replicating sequence, phage, or linear or circular single-stranded or double-stranded DNA or RNA nucleotide sequence, derived from any source, capable of genomic integration or autonomous replication, comprising a nucleic acid molecule in which one or more sequences have been linked in a functionally operative manner. Such recombinant constructs or vectors are capable of introducing a 5′ regulatory sequence or promoter region and a DNA sequence for a selected gene product into a cell in such a manner that the DNA sequence is transcribed into a functional mRNA, which may or may not be translated and therefore expressed.

“Restriction enzyme” refers to an enzyme that recognizes a specific sequence of nucleotides in double stranded DNA and cleaves both strands; also called a restriction endonuclease. Cleavage typically occurs within the restriction site or close to it.

“Selectable marker” refers to a nucleic acid sequence whose expression confers a phenotype facilitating identification of cells containing the nucleic acid sequence. Selectable markers include those, which confer resistance to toxic chemicals (e.g. ampicillin, kanamycin) or complement a nutritional deficiency (e.g. uracil, histidine, leucine).

“Screenable marker” refers to a nucleic acid sequence whose expression imparts a visually distinguishing characteristic (e.g. color changes, fluorescence).

“Transcription” refers to the process of producing an RNA copy from a DNA template.

“Transformation” refers to a process of introducing an exogenous nucleic acid sequence (e.g., a vector, plasmid, or recombinant nucleic acid molecule) into a cell in which that exogenous nucleic acid is incorporated into a chromosome or is capable of autonomous replication. A cell that has undergone transformation, or a descendant of such a cell, is “transformed” or “recombinant.”

“Translation” refers to the production of protein from messenger RNA.

“Unit” of enzyme refers to the enzymatic activity and indicates the amount of micromoles of substrate converted per mg of total cell proteins per minute.

“Vector” refers to a DNA or RNA molecule (such as a plasmid, cosmid, bacteriophage, yeast artificial chromosome, or virus, among others) that carries nucleic acid sequences into a host cell. The vector or a portion of it can be inserted into the genome of the host cell.

The term “yield” refers to the amount of industrial product or L-ascorbic acid produced by the recombinant organism, as (molar or weight/volume) divided by the amount of precursor consumed (molar or weight/volume) multiplied by 100.

List of Abbreviations:

Asc L-ascorbic acid (vitamin C)

AGD L-galactono-1,4-lactone dehydrogenase (without signaling peptide)

ALO D-arabinono-1,4-lactone oxidase

ARA D-arabinose dehydrogenase

Gal L-galactono-1,4-lactone

Gul L-gulono-1,4-lactone

LGDH L-galactose dehydrogenase

ME Mannose epimerase

MIP Myoinositol phosphatase

RGLO L-gulono-1,4-lactone oxidase

TCA trichloroacetic acid

TPI triosephosphateisomerase

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Materials and Methods

1. Determination of Ascorbic Acid

Ascorbic acid was determined spectrophotometrically following the method of Sullivan, M. X. et al. (1955), Assoc. Off. Agr. Chem., 38, 514-518). The sample (135 μl) was mixed in a cuvette with 40 μl of H₃PO₄ (85%). Then 675 μl of α,α′-Bipyridyl (0.5%) and 135 μl FeCl₃ (1%) were added. After 10 min the absorbance at 525 nm was measured. In some experiments, the identity of the ascorbic acid was confirmed by HPLC (Tracer Extrasil Column C8, 5 μM, 15×0.46 cm, Teknokroma, S. Coop. C. Ltda. # TR-016077; Eluent: 5 mM cetyltrimethylammonium bromide, 50 mM KH₂PO₄ in 95/5H₂O/Acetonitrile; Flow rate: 1 ml min⁻¹, Detection UV @ 254 nm) with pure L-ascorbic acid (Aldrich, A9,290-2) as standard.

2. Amplification of Specific Gene Sequences

To amplify specific gene sequences, PfuTurbo DNA polymerase (Stratagene #600252) was used on a GeneAmp PCR System 9700 (PE Appl. Biosystems, Inc.). Standard conditions used were: 400 μM dNTP, 0.5 μM primers, 0.5 mM MgCl₂ (in addition to the buffer), and 3.75 U Pfu per 100 μl reaction.

The sequences of the genes used have been publicly reported via Genbank, as follows, except for MIP. The MIP sequence listed as SEQ ID NO:4 differed from the Genbank sequence, accession no. NM_{—}111155, by two translationally silent point substitutions: at bp271, A (NM_{—}111155) to T (SEQ ID NO:4); at bp 685, T (NM_{—}11155) to G (SEQ ID NO:4).

| Gene | Genbank accession no(s). | SEQ ID NO: |
|---|---|---|
| ME | AY116953 | 3 |
| MIP | n.a. | 4 |
| ALO | U40390, AB009401 | 5, 6 |
| LGDH | | 7 |

The following program was used for amplification of ALO:

| | | | |
|---|---|---|---|
| 94° C. | 5 min | | |
| 94° C. | 45 s | | |
| 50° C. | 30 s | {close oversize brace} | 33 cycles |
| 72° C. | 1 min 40 s | | |
| 72° C. | 7 min | | |
| 4° C. | To | | |
| | completion | | |

The following program was used for amplification of LGDH:

| | | | |
|---|---|---|---|
| 94° C. | 5 min | | |
| 94° C. | 45 s | | |
| 56° C. | 30 s | {close oversize brace} | 33 cycles |
| 72° C. | 1 min 40 s | | |
| 72° C. | 7 min | | |
| 4° C. | To | | |
| | completion | | |

The following program was used for amplification of ME:

| | | | |
|---|---|---|---|
| 94° C. | 5 min | | |
| 94° C. | 15 s | | |
| 50° C. | 30 s | {close oversize brace} | 30 cycles |
| 72° C. | 1 min 30 s | | |
| 72° C. | 7 min | | |
| 4° C. | To | | |
| | completion | | |

The following program was used for amplification of MIP:

| | | | |
|---|---|---|---|
| 94° C. | 5 min | | |
| 94° C. | 15 s | | |
| 59.8° C. | 30 s | {close oversize brace} | 28 cycles |
| 72° C. | 45 s | | |
| 72° C. | 7 min | | |
| 4° C. | To | | |
| | completion | | |

Template DNA for LGDH, ME, and MIP: 50 ng plasmid cDNA library pFL61 Arabidopsis (ATCC #77500 (Minet M. et al. (1992), Plant J. 2, 417-422)). Template DNA for ALO: 50 ng genomic DNA from S. cerevisiae GRF18U, extracted using a standard method. PCR products were blunt-end cloned into the EcoRV site of pSTBlue-1 using the perfectly blunt cloning kit from Novagen Inc. (#70191-4).

| | Gene |
|---|---|
| Oligonucleotides used | amplified |
| SEQ ID NO:8: tttcaccatatgtctactatcc | ALO |
| SEQ ID NO:9: aaggatcctagtcggacaactc | (yeast) |
| SEQ ID NO:10: atgacgaaaatagagcttcgagc | LGDH |
| SEQ ID NO:11: ttagttctgatggattccacttgg | (plant) |
| SEQ ID NO:12: gcgccatgggaactaccaatggaaca | ME |
| SEQ ID NO:13: gcgctcgagtcactcttttccatca | (plant) |
| SEQ ID NO:14: atccatggcggacaatgattctc | MIP |
| SEQ ID NO:15: aatcatgcccctgtaagccgc | (plant) |

3. Plasmid Construction

The naming convention used herein is that pSTBlue-1 containing, for example, ALO in the sense direction regarding its multiple cloning site (MCS) was designated pSTB ALO-1. In a further example, pSTBlue-1 containing ALO in the antisense direction regarding its MCS was designated pSTB ALO-2, and so on.

Inserts were cloned using either the pYX series (R&D Systems, Inc.) or the centromeric expression plasmids pZ₃ and pZ₄ (P. Branduardi, et al. The Yeast Zygosaccharomyces bailii: a New Host for Heterologous Protein Production, Secretion and for Metabolic Engineering Applications, FEBS Yeast Research, FEMS Yeast Res. (2004) 4, 493-504). Standard procedures were employed for all cloning purposes, (Sambrook, J. and Russell, D. W., Molecular Cloning: A Laboratory Manual, 3^{rd} Edition, Cold Spring Harbor Laboratory Press, 2001).

| | | | | |
|---|---|---|---|---|
| pSTB LGDH-1 | EcoRI | pYX022 | pH LGDH | HIS 3 (marker) |
| pSTB ALO-1 | EcoRI | pYX042 | pL ALO | LEU 2 (marker) |
| pSTB ME-1 | EcoRI | pZ₃ | pZ₃ ME | Kan^{r} (marker) |
| pSTB ME-1 | EcoRI | pZ₄ | PZ₄ ME | Hph^{r} (marker) |
| pSTB MIP-1 | EcoRI | pYX012 | pU MIP | URA 3 (marker) |

For all the work performed below, the yeast control strains were transformed with the corresponding empty vectors.

4. Yeast Cultivation and Examination:

Yeast strains used were S. cerevisiae GRF18U (Brambilla, L. et al., 1999, FEMS Microb. Lett. 171, 133-140), S. cerevisiae GRFc (Brambilla et al. 1999 FEMS Microb. Lett. 171: 133-140), S. cerevisiae BY4742 (MATα; his3; leu2, lys2; ura3, EuroScarf Accession No. Y10000), S. cerevisiae YML007w (BY4742; MATα; his3; leu2, lys2; ura; YML007w::KanMX4 (the yap1 deleted strain) EuroScarf Accession No. Y10569); CEN.PK 113-5D (MATα ura3-52; cir+) (see, for example, VanDijken et al. (2000) Enzyme Microb. Technol. 26, 706-714); and BY4741 (MATα; his3; leu2; met 15; ura3, Euroscarf Accession No. Y00000), or strains derived from them through transformation with the different developed plasmids. All strains were cultivated in shake flasks in minimal medium (0.67% w/v YNB (Difco Laboratories, Detroit, Mich. #919-15), 2% w/v glucose or mannose, with addition of the appropriate amino acids or adenine or uracil, respectively, to 50 μL-¹) and/or the appropriate antibiotic (G418 or hygromicin to 500 mg/l and 400 mg/l, respectively) under standard conditions (shaking at 30° C.). The initial optical density at 660 nm was about 0.05 for ascorbic acid determination, and 0.1 for the kinetics of the recovery from oxidative stress.

Cells were recovered by centrifugation at 4000 rpm for 5 min at 4° C., washed once with cold distilled H₂O, and treated as follows: for determination of intracellular ascorbic acid, cells were resuspended in about 3 times the pellet volume of cold 10% TCA, vortexed vigorously, kept on ice for about 20 min, and then the supernatant was cleared from the cell debris by centrifugation.

5. Yeast Transformation:

Transformation of yeast cells was performed by the standard LiAc/ss-DNA/PEG method (Gietz, R. D. and Schiestl, R. H. (1996), Transforming Yeast with DNA, Methods in Mol. and Cell. Biol.).

Experimental Results

6. Expression of Arabidopsis thaliana ME, MIP, LDGH and S. cerevisiae ALO in GRF18U

The genes encoding A. thaliana ME, S. cerevisiae ALO, A. thaliana LGDH, and A. thaliana MIP were placed under the control of the TPI (triosephosphateisomerase) promoter each on its own integrative plasmid, except ME, which was sub-cloned in a centromeric plasmid. Two or more of the genes were integrated into S. cerevisiae GRF18U and BY4742. Each gene was integrated at a unique locus.

FIG. 1 provides a schematic representation of the current understanding of the physiological biosynthetic pathway leading from D-glucose to L-ascorbic acid in plants. The following enzymes are involved: A, L-galactono-1,4-lactone dehydrogenase (1.3.2.3), B, L-galactose dehydrogenase, C, myoinositol phosphatase (3.1.3.23), D, pyrophosporylase, E, GDP-mannose-3,5-epimerase (5.1.3.18), F, mannose-1-phosphate guanylyltransferase (2.7.7.22), G, phosphomannomutase (5.4.2.8), H, mannose-6-phosphate isomerase (5.3.1.8), I, glucose-6-phosphate isomerase (5.3.1.9), J; hexokinase (2.7.1.1).

In the pathway shown in FIG. 1, ALO catalyzes reaction A, LGDH catalyzes reaction B, ME catalyzes reaction E, and MIP catalyzes reaction C.

Wild-type yeast cells are known to produce GDP-mannose (reactions F-J in FIG. 1) and to transport it to the endoplasmic reticulum.

The table below shows the conversion of D-Glucose and D-Mannose to ascorbic acid by S. cerevisiae GRFc (control), or S. cerevisiae GRF18U transformed with (i) ALO and LDGH; (ii) ALO, LDGH and ME; or (iii) ALO, LDGH, ME and MIP. Cells were grown on mineral medium (2% glucose or mannose, 0.67% YNB) starting from an OD⁶⁶⁰ of 0.05. After 24 hours of growth, ascorbic acid was determined. While both the wild-type GRFc and GRF18U cells transformed with ALO and LGDH did not accumulate ascorbic acid, cells transformed with ALO, LDGH and ME, or ALO, LDGH, ME and MIP, respectively unexpectedly accumulated considerable amounts (i.e. greater than background levels) of ascorbic acid.

Transformed yeast were batch grown on glucose- or mannose-based media:

| | Total (ascorbate | Total (ascorbate plus |
|---|---|---|
| | plus erythroascorbate) | erythroascorbate) on |
| | on glucose-containing | mannose-containing |
| Expressed gene | media | media |
| Wt (control) | 0.0205 | 0.0220 |
| ALO, LGDH (control) | 0.0210 | 0.0221 |
| ALO, LDGH, ME | 0.0302 | 0.0332 |
| ALO, LDGH, ME, MIP | 0.0450 | 0.0296 |

(Total (ascorbate plus erythroascorbate) values are mg/OD⁶⁶⁰ of Biomass/L)

The values determined in the control strain indicate the production of erythroascorbate normally produced by wild type yeasts.

We conclude that the yeast endogenously possesses activities which can nonspecifically catalyze reactions from GDP-L-galactose to L-galactose (see FIG. 1). Specifically, though not to be bound by theory, we conclude that GDP-L-galactose spontaneously hydrolyses to L-galactose-1-P and that a nonspecific phosphatase catalyzed the conversion of L-galactose-1-P to L-galactose, which was then converted to L-ascorbic acid by LGDH and ALO. MIP provided superior catalysis of L-galactose-1-P to L-galactose than did the putative nonspecific phosphatase (ALO, LGDH, ME, MIP vs. ALO, LGDH, ME).

We did not observe any ascorbic acid accumulation in the medium.

7. Sensitivity to Oxidative Stress

FIG. 2 shows that YML007w yeast hosts are particularly sensitive to oxidative stress. Yap1p activates genes required for the response to oxidative stress; deletion of this gene leads to the observed phenotype (Rodrigues-Pousada C A, et al. (2004) FEBS Lett.

567, 80-85)

The following yeast strains have been analyzed:

BY4742 (▴).

YML007w (◯)

FIG. 2A. The yeast strains were grown on mineral medium (2% glucose, 0.67% YNB) starting from an OD⁶⁶⁰ of 0.1.

FIG. 2B. The yeast strains were grown on mineral medium (2% glucose, 0.67% YNB) starting from an OD⁶⁶⁰ of 0.1 in the presence of 0.8 mM of H₂O₂.

FIG. 2C. The yeast strains were grown on mineral medium (2% glucose, 0.67% YNB) starting from an OD⁶⁶⁰ of 0.1 in the presence of 1.0 mM of H₂O₂.

The two strains grew in the absence of H₂O₂ (FIG. 2A) while growth of the YML007w yeast host was strongly delayed in medium containing 0.8 mM of hydrogen peroxide (FIG. 2B) and completely impaired in the medium containing 1 mM of hydrogen peroxide (FIG. 2C).

8. Effect of Ascorbic Acid in Media on Stress Tolerance

FIG. 3 shows that the growth sensitivity of YML007w yeast, as shown in FIG. 2, can be rescued by adding ascorbic acid to the medium, and that the effect of ascorbic acid in the medium on robustness is concentration dependent and can be optimized for different yeast strains.

FIG. 3A. The yeast strains were grown on minimal medium (2% glucose, 0.67% YNB) starting from an OD⁶⁶⁰ of 0.1 in presence of 0.8 mM of H₂O₂. Ascorbic acid was added at T=0 at a final concentration of 15 mg/L. BY4742 (▴); YML007w (◯).

FIG. 3B. The yeast strains were grown on mineral medium (2% glucose, 0.67% YNB) starting from an OD⁶⁶⁰ of 0.1 in presence of 1.0 mM of H₂O₂. Ascorbic acid was added at T=0 at a final concentration of 15 mg/L. BY4742 (▴); YML007w (◯).

FIG. 3C. Three yeast strains (GRFc, BY4741, and CEN.PK 113-5D) were grown in 2×YNB medium (2% glucose, 1.34% YNB), containing lactic acid at 40 g/l, pH3. Ascorbic acid was added to the medium at the concentrations shown. The data demonstrate that the negative effects of lactic acid on growth can be overcome by exogenous ascorbic acid, and that the effect of ascorbic acid is dose dependent.

9. Effect of Endogenous Ascorbic Acid on Sensitivity to Oxidative Stress

FIG. 4 shows that the growth defects of the YML007w yeast hosts can be rescued following expression of ALO, LDGH, ME, and MIP.

The following yeast strains have been analyzed:

BY4742 (▴)

YML007w expressing ALO, LDGH and ME (□)

YML007w expressing ALO, LDGH, ME and MIP (▪)

FIG. 4A. The yeast strains were grown on minimal medium (2% glucose, 0.67% YNB) starting from an OD⁶⁶⁰ of 0.1 in presence of 0.8 mM of H₂O₂.

FIG. 4B. The yeast strains were grown on minimal medium (2% glucose, 0.67% YNB) starting from an OD⁶⁶⁰ of 0.1 in presence of 1.0 mM of H₂O₂.

Endogenous production of ascorbic acid “rescued” the yeast from stress-induced growth inhibition in a manner similar to that obtained by adding ascorbic acid to the culture medium (see FIG. 3).

10. Effect of Endogenous Ascorbic Acid on Robustness of GRF Yeast Strains

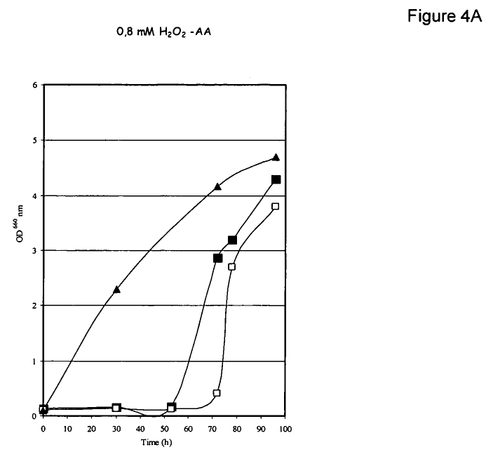

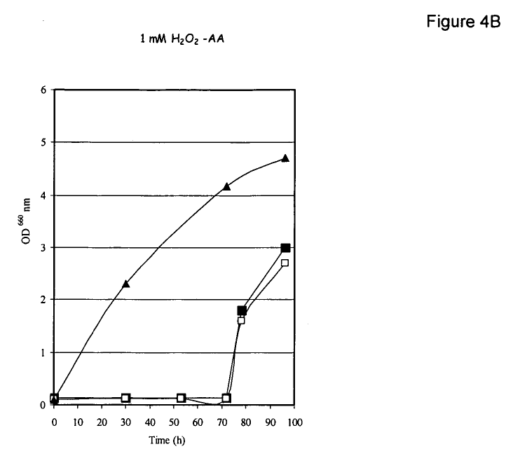

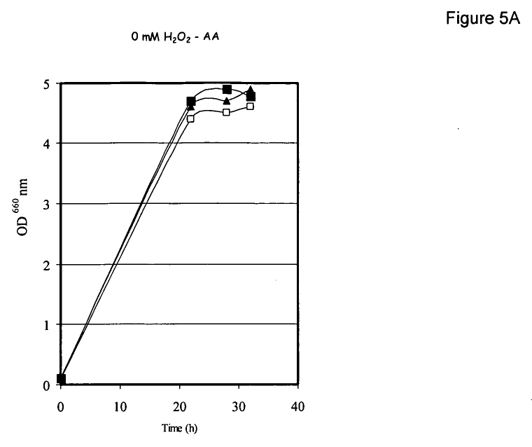

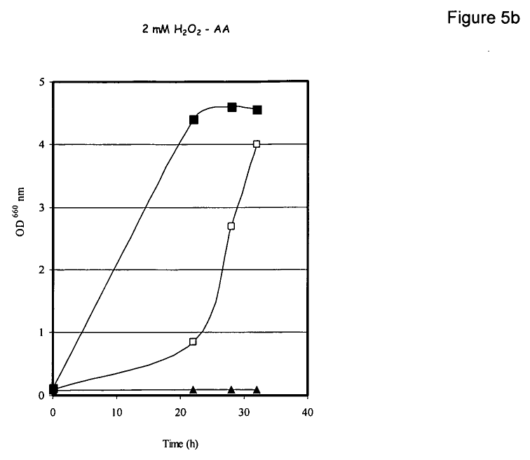

FIG. 5 shows that the wild type GRF yeast strain is sensitive to fermentative stress conditions (stress condition induced by adding 2 mM of H₂O₂); surprisingly, the recombinant yeast strains producing ascorbic acid show a strong robustness, indicating an increased tolerance to stress. The following yeast strains were analyzed: GRFc (closed triangle); GRF18U expressing ALO, LDGH and ME (open square); and GRF18U expressing ALO, LDGH, ME and MIP (closed square).

FIG. 5A. The yeast strains were grown on mineral medium (2% glucose, 0.67% YNB) starting from an OD660 of 0.1.

FIG. 5B. The yeast strains were grown on mineral medium (2% glucose, 0.67% YNB) starting from an OD660 of 0.1 in presence of 2.0 mM of H₂O₂. The wild type strain does not consume glucose.

All the strains used in this experiment bear the same auxotrophic complementation and the same antibiotic resistance cassettes (that are necessary for the expression of the different heterologous genes), so that it was possible to use the same media for all of them, either the ones expressing 3 or 4 heterologous genes or the wild type strain.

For this experiment, as a classical example of stress, we challenged wild type yeast cells with H₂O₂. As expected, wild type cells grow well in the absence of H₂O₂ (FIG. 5A), but the same yeast cells do not grow in the presence of the H₂O₂ (FIG. 5B). It is generally accepted that this external stressor leads to damage to DNA, damage to lipids, damage to proteins, and damage to membranes, among other subcellular structures, and ultimately leads to a loss of cell viability and cell integrity. Therefore, it is not surprising that the presence of this stressor leads to zero production, zero productivity and zero yield of the product (in this case, wild type yeast biomass), as shown in FIG. 5B.

By the transformation of wild type GRF yeast with (i) LGDH, ALO, and ME or (ii) LGDH, ALO, ME and MIP, the recombinant yeast produced ascorbic acid, as described above, whereas wild type yeasts do not naturally produce ascorbic acid. Surprisingly, the bioprocess based on these recombinant yeasts showed a high production, high productivity, and a high yield of the product, yeast biomass (FIG. 5B). Values for production, productivity, and yield are greater than 0.00 in the recombinant yeast (values for the control strain).

This experiment shows the two recombinant GRF yeast strains are more tolerant to stress than wild type GRF yeast, and may therefore be more suitable for certain industrial processes. Though not to be bound by a single theory, we consider it likely the recombinant yeast are less sensitive to diverse stressors, possibly through both direct scavenging of reactive oxygen species (ROS) by ascorbic acid and interference by ascorbic acid with unwanted stress reactions, such as apoptosis, cell death, viability loss, and loss of cell integrity.

11. Effect of Endogenous Ascorbic Acid on ROS and Viability

The S. cerevisiae strains YML007w and YML007w transformed to express ALO, LDGH, ME, and MIP were grown in minimal glucose medium with or without addition of H₂O₂. Each culture was then split into two, and one was stained with dehydrorodamine for the detection of reactive oxygen species (ROS), the other was stained with propidium iodide for viability determination. Samples were then analyzed with a flow cytometer and compared. FIG. 6 demonstrates a correlation between ascorbic acid production and reduction in ROS formation, as well as reduction of the fraction of nonviable cells.

12. Effect of Endogenous Ascorbic Acid on Sensitivity to Low pH and Lactic Acid

The S. cerevisiae strains BY4742c and BY4742 transformed to express ALO, LDGH, ME, MIP were inoculated in minimal glucose medium, minimal glucose medium at low pH (2.2), or minimal glucose medium at pH 3.0 containing 38 g/l of lactic acid. FIG. 7 shows growth curves for BY4742c (open squares) and the same yeast background transformed to produce ascorbic acid (dark squares) in minimal glucose medium, pH 2.2 (FIG. 7a), and in minimal glucose medium containing 38 g/l lactic acid, pH 3.0 (FIG. 7b). In the transformed yeast strain producing ascorbic acid, peak levels of cells at low pH are approximately three-fold greater and peak levels of cells in medium containing lactic acid are approximately five-fold greater compared with the non-transformed strain.

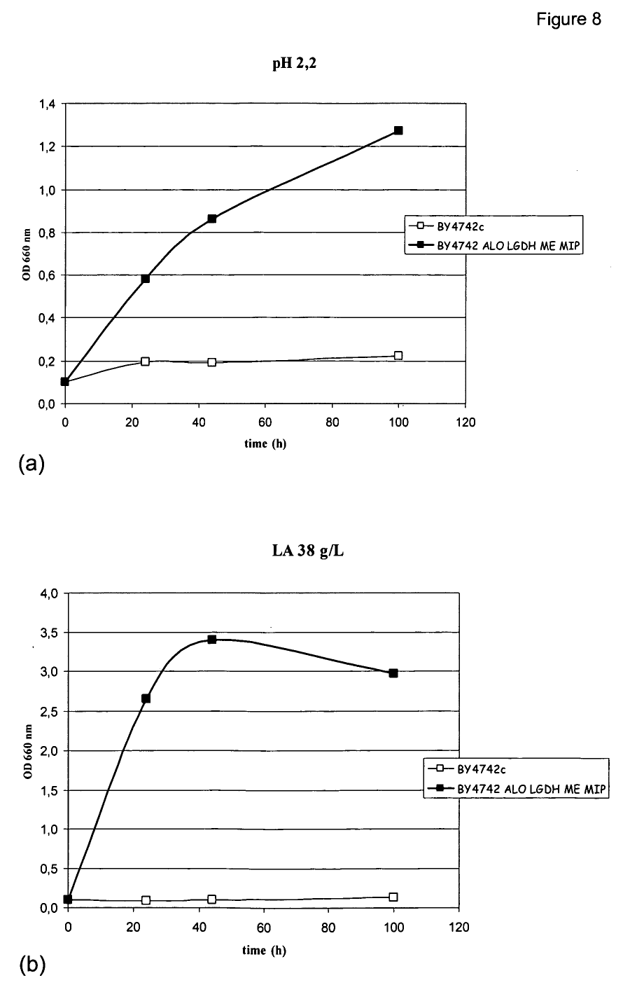

The same experiment was conducted after the two yeast strains were grown for about 24 hours in minimal glucose medium and then inoculated in minimal glucose medium at low pH (2.2), or minimal glucose medium at pH 3.0 containing 38 g/l of lactic acid. The results are shown in FIG. 8. At low pH, the transformed strain producing lactic acid showed more than a six-fold increase in peak cell numbers compared with the non-transformed strain (FIG. 8a). In medium containing lactic acid, the non-transformed strain showed no increase in growth, whereas the transformed yeast strain producing ascorbic acid showed exponentional growth with an approximately 3.5 fold increase at peak levels (FIG. 8b).

13. Effect of Exogenous Ascorbic Acid on Growth of Lactic Acid Producing Yeast m850.

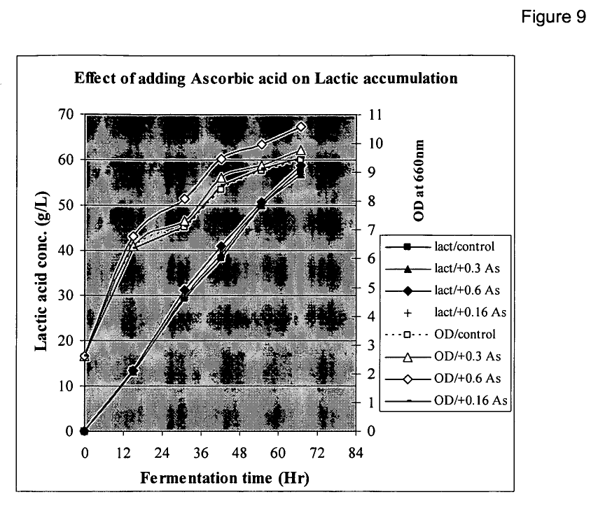

S. cerevisiae strain NRRL Y-30696 was inoculated in minimal glucose medium and 2.78 g/L CaCO₃ or minimal glucose medium with 2.78 g/L CaCO₃ and 0.16, 0.3, or 0.6 g/L ascorbic acid. OD660 (open symbols) and lactic acid (closed symbols) were monitored with time. The pH dropped in each case to 2.5 at 67 hours. FIG. 9 shows that growth, as measure by OD660, increased with increasing ascorbic acid, 0 g/L (O), 0.16 g/L (+), 0.3 g/L (▴), or 0.6 g/L (♦), while lactic acid production was equivalent at each level.

14. Construction of a Yeast Strain Co-Producing Lactic Acid and Ascorbic Acid.

S. cerevisiae NRRL Y-30696 (Y-30696) has previously been engineered to produce lactic acid. The ability to co-produce a low level of endogenous ascorbic acid could be introduced by integrating the genes required for ascorbic acid production into Y-30696. As shown above, production of significant endogenous L-ascorbic acid can be achieved by the expression of sequences encoding ME, LGDH, and ALO+/−MIP. One or more of these genes, functionally coupled to an appropriate promoter, could be added to the L-LDH bearing plasmid of Y-30696, while additional genes, coupled to appropriate promoters, could be introduced at the sites of the deleted PDC genes. Methods for these steps are known in the art, and are found in Sauer, M., et al. (2004) Applied Environmental Microbiology 70, 6086-6091.

While the compositions and methods and yeast strains of this invention have been described in terms of particular embodiments, it will be apparent to those of skill in the art that variations may be applied without departing from the concept, spirit and scope of the invention.

### REFERENCES

References which are specifically incorporated herein by reference are so marked.
1) Alberts, B., et al. 2002, Molecular Biology of the Cell, 4^{th} Edition, New York: Garland Science Publ.
2) Bauw, G. J. C., Davey, M. W., Østergaard, J., Van Montagu, M. C. E., 1998, Production of Ascorbic Acid in Plants, 1998, International Patent Application, WO98/50558
3) Becker D. M. and Guarente L. 1991, Methods in Enzymology, 194:18
4) Berry, A., Running, J., Severson, D. K., Burlingame, R. P., 1999, Vitamin C Production in Microorganisms and Plants, International Patent Application, WO99/64618
5) Bizukojc, M. and Ledakowicz, S. 2004, Process Biochemistry 39, 2261-2268
6) Bleeg, H. S., Christensen, F., 1982, Biosynthesis of Ascorbate in Yeast, Purification of L-Galactono-1,4-lactone Oxidase with Properties Different from Mammalian L-Gulonolactone Oxidase, Eur. J. Biochem. 127, 391-96
7) Bosman, F., et al. (2006) U.S. Pat. No. 7,048,930
8) Brambilla, L., Bolzani, D., Compagno, C., Carrera, D., van Dijken, J. P., Pronk, J. T., Ranzi, B. M., Alberghina, L., Porro, D. 1999, NADH reoxidation does not control glycolytic flux during exposure of respiring Saccharomyces cerevisiae cultures to glucose excess, FEMS Microb. Lett. 171, 133-140
9) Branduardi, P., et al. 2006, International Specialised Symposium on Yeast. ISSY25, Systems Biology of Yeast—From Models to Applications. “L-ascorbic acid production from D-glucose in metaboloic engineered Saccharomyces cerevisiae and its effect on strain robustness.” Hanasaari, Espoo, Finland, Jun. 21, 2006
10) Close, D. C., et al. 2006, Oxidative Stress, Exercise, and Aging, H. M. Alessio, A. E. Hagerman, Eds., pp. 9-23
11) Conklin, P. L., et al. (2006) Arabidopsis thaliana VTC4 encodes L-galactose-1-P phosphatase, a plant ascorbic acid biosynthetic enzyme, J. Biol. Chem. 281, 15662-70
12) Dunn-Coleman, N. S., et al. 2006, U.S. Pat. No. 7,074,608
13) Durrens P. et al. 1990, Curr. Genet., 18:7
14) Foti, D. M., et al. 1999, J. Biol. Chem. 274, 30402-30409
15) Geertman, J. M, et al. 2006, Metabolic Engineering, June 30:(Epublication)
16) Gerard, G. F., et al. 2006, U.S. Pat. No. 7,115,406
17) Gietz, R. D. and Schiestl, R. H., 1996, Transforming Yeast with DNA, Methods in Mol. and Cell. Biol. 5 (5) 255-269. This article is incorporated herein by reference.
18) Hancock, R. D., Galpin, J. R., and Viola, R. 2000, Biosynthesis of L-ascorbic acid (vitamin C) by Saccharomyces cerevisiae. FEMS Microbiol. Lett. 186, 245-250
19) Harris, L. M., et al. 2001, Journal of Industrial Microbiology & Biotechnology 27, 322-328
20) Ho, N. W. Y. and Tsao, G. T. 1998, U.S. Pat. No. 5,789,210
21) Huh, W. K., Kim, S. T., Yang, K. S., Seok, Y. J., Hah, Y. C., Kang, S. O., 1994, Characterisation of D-arabinono-1,4-lactone oxidase from Candida albicans ATCC 10231, Eur. J. Biochem. 225, 1073-1079
22) Huh, W. K., Lee, B. H., Kim, S. T., Kim, Y. R., Rhie, G. E., Baek, Y. W., Hwang, C. S., Lee, S. J., Kang, S. O., 1998, D-Erythroascorbic acid is an important antioxidant molecule in S. cerevisiae, Mol. Microb. 30, 4, 895-903
23) Ingram, L. O. and Zhou, S. WO2000/071729
24) Ito H. et al. ((1983), J. Bacteriol., 153:163
25) Kim, S. T., Huh, W. K., Kim, J. Y., Hwang, S. W., Kang, S. O., 1996, D-Arabinose dehydrogenase and biosynthesis of erythroascorbic acid in Candida albicans, BBA 1297, 1-8
26) Kim, S. T., Huh, W. K., Lee, B. H., Kang, S. O., 1998, D-Arabinose dehydrogenase and its gene from Saccharomyces cerevisiae, BBA 1429, 29-39
27) Knapp, S., et al. 1992, U.S. Pat. No. 5,159,062
28) Kreger-van Rij, N. J. W., “The Yeasts,” Vol. 1 of Biology of Yeasts, Ch. 2, A. H. Rose and J. S. Harrison, Eds. Academic Press, London, 1987. This article is incorporated herein by reference.
29) Kumar, M. 2000 Production of ascorbic acid using yeast, International patent application WO 00/34502
30) Lee, B. H., Huh, W. K., Kim, S. T., Lee, J. S., Kang, S. O., 1999, Bacterial Production of D-Erythroascorbic Acid and L-ascorbic acid through Functional Expression of Saccharomyces cerevisiae D-Arabinono-1,4-Lactone Oxidase in Escherichia coli, App. Env. Microb. 65, 10, 4685-4687
31) Lee, S. Y., et al. 2003, Lett. Appl. Microbiol. 36, 121-128.
32) Lowry, O. H., Rosebrough, N. J., Farr, A. L., Randall, R. J., 1951, Protein Measurement with the Folin Phenol Reagent, J. Biol. Chem. 193, 265-275
33) Minet, M., Dufour, M. E., Lacroute, F., 1992, Plant J., 2, 417-422. This article is incorporated herein by reference.
34) Mongkolsuk, S. and Helmann, J. D. 2002, Molecular Microbiology 45, 9-15
35) C. Moraitis and B. P. G. Curran. 2004, Reactive oxygen species may influence the heat shock response and stress tolerance in the yeast Saccharomyces cerevisiae, Yeast 21, 313-323
36) Nishikimi, M., Noguchi, E., Yagi, K., 1978, Occurrence in Yeast of L-Galactonolactone Oxidase Which is Similar to a Key Enzyme for Ascorbic Acid Biosynthesis in Animals, L-Gulonolactone Oxidase, Arch. Biochem. Biophys. 191, 2, 479-486
37) Østergaard, J., Persiau, G., Davey, M. W., Bauw, G., Van Montagu, M., 1997, Isolation of a cDNA Coding for L-Galactono-γ-Lactone Dehydrogenase, an Enzyme involved in the Biosynthesis of Ascorbic Acid in Plants, J. Biol. Chem. 272, 48, 30009-30016
38) Padh H. 1990, Cellular functions of ascorbic acid, Biochem. Cell Biol. 68, 1166-1173.
39) Pizza, M., et al. 2006, U.S. Pat. No. 7,115,730
40) Porro, D., et al., U.S. Pat. No. 6,429,006. This patent is incorporated herein by reference.
41) Porro, D., et al., U.S. Pat. No. 6,630,330. This patent is incorporated herein by reference.
42) Porro, D., et al., U.S. Pat. No. 7,049,108. This patent is incorporated herein by reference.
43) Reichstein, T. 1941, U.S. Pat. No. 2,265,121
44) Rodrigues-Pousada C A, et al. 2004, FEBS Lett. 567, 80-85
45) Roland, J. F., Cayle, T., Dinwoodie, R. C., Mehnert, D. W., 1986, Fermentation Production of Ascorbic Acid from L-Galactonic Substrate, U.S. Pat. No. 4,595,659
46) Roland, J. F., Cayle, T., Dinwoodie, R. C., Mehnert, D. W., 1990, Bioconversion Production of Ascorbic Acid with L-Galactono-1,4-Oxidase, U.S. Pat. No. 4,916,068
47) Sambrook, J. and Russell, D. W., Molecular Cloning: A Laboratory Manual, 3^{rd} Edition, Cold Spring Harbor Laboratory Press, 2001. This book is incorporated herein by reference.
48) Santos, et al. 2006, Abstracts of Papers, 232nd ACS National Meeting, San Francisco, Calif., United States, Sep. 10-14, 2006, BIOT-243
49) Sauer, M., et al. 2004, Applied Environmental Microbiology 70, 6086-6091
50) Shiuan, D., US2003/0104584
51) Smirnoff, N., Wheeler, G., 1999, Plant Galactose Dehydrogenase, International Patent Application, WO99/33995
52) Spencer J. F. et al. 1988, Journal of Basic Microbiology 28, 321-333
53) Sugiyama, K., et al. 2000, J Biol. Chem. 275, 15535-15540
54) Sullivan, M. X., Clarke, H. C. N., 1955, A highly specific procedure for ascorbic acid, Assoc. Off. Agr. Chem. 38, 2, 514-518. This article is incorporated herein by reference.
55) Tiffany, B. et al. 2004, Nucleic Acids Research 32, 3712-3723
56) Valpuesta, V. and Botella, M. A. 2004, Biosynthesis of L-ascorbic acid in plants: new pathways for an old antioxidant, Trends in Plant Science 12, 573-577
57) VanDijken et al. 2000, Enzyme Microb. Technol. 26, 706-714
58) Wang, L., et al. 2003, Biotechnology Advances 23, 115-129
59) Wésolowski-Louvel, M., Prior, C., Bornecque, D., Fukuhara, H. 1992, Rag-mutations involved in glucose metabolism in yeast: isolation and genetic characterization. Yeast 8, 711-719
60) Wheeler, G. L., Jones, M. A., Smirnoff, N., 1998, The biosynthetic pathway of vitamin C in higher plants, Nature 393, 365-368
61) WO2006/083410
62) WO2005/090589
63) Xiao, A. et al. 2006, Improvement of cell viability and hirudin production by ascorbic acid in Pichia pastoris fermentation, Appl. Microbiol. Biotechnol. 72, 837-844

What is claimed is:

1. A method of increasing stress tolerance in a recombinant organism that is engineered for industrial production of at least one product comprising functionally transforming the recombinant organism with a coding region encoding a mannose epimerase (ME), a coding region encoding an L-galactose dehydrogenase (LGDH), and a coding region encoding a D-arabinono-1,4-lactone oxidase (ALO), whereby the recombinant organism is enabled to produce ascorbic acid endogenously.

2. The method of claim 1, wherein the recombinant organism is further functionally transformed with a coding region encoding a myoinositol phosphatase (MIP).

3. The method of claim 1, wherein the recombinant organism is further functionally transformed with a coding region encoding an enzyme selected from the group consisting of L-galactono-1,4-lactone dehydrogenase (AGD), D-arabinose dehydrogenase (ARA), and L-gulono-1,4-lactone oxidase (GLO).

4. The method of claim 1, wherein the recombinant organism produces lactic acid.

5. The method of claim 1, wherein the recombinant organism is an organism selected from the group consisting of bacteria, yeast, filamentous fungi, and animal cells.

6. The method of claim 1, wherein the recombinant organism is a yeast belonging to a genus selected from the group consisting of Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces.

7. The method of claim 5, wherein the recombinant organism is a yeast selected from the group consisting of S. cerevisiae strain GRF18U; S. cerevisiae strains W3031B, BY4741, BY4742, CEN.PK 113-5D and YML007w; K. lactis strain CBS2359; Z. bailii strain ATCC 60483; S. cerevisiae strains NRRL Y-30696, NRRL Y-30698, NRRL Y-30742; K. lactis strains PM6-7/pEPL2, PMI/C1[pELP2]; Z. bailii strains ATTC36947/pLAT-ADH, ATCC60483/pLAT-ADH.

8. The method of claim 5, wherein the recombinant organism is a bacterium of a genus selected from the group consisting of Bacillus, Escherichia, Lactobacillus, Lactococcus, Pseudomonas, and Acetobacter.

9. The method of claim 5, wherein the recombinant organism is a bacterium selected from the group of bacterial strains producing lactic acid consisting of Bacillus coagulans, Lactobacillus helveticus, Lactobacillus delbrueckii, Lactobacillus casei, Lactobacillus acidophilus, Lactobacillus bulgaricus, Lactobacillus pentosus, and Streptococcus thermophilus.

10. The method of claim 5, wherein the recombinant organism is a filamentous fungus of a genus selected from the group consisting of Aspergillis, Rhizopus, and Trichoderma.

11. The method of claim 5, wherein the recombinant organism is a filamentous fungus selected from the group consisting of Aspergillus kawachii, Aspergillus nidulans, Aspergillus niger, Aspergillus oryzae, Rhizopus arrhizus, Rhizopus microsporus, Rhizopus oryzae, Trichoderma harzianum, Trichoderma reesei, and Trichoderma viride.

12. The method of claim 1, wherein the ME has at least about 95% identity with SEQ ID NO:1.

13. The method of claim 2, wherein the MIP has at least about 95% identity with SEQ ID NO:2.

14. The method of claim 1, wherein the recombinant organism is a yeast, and wherein the yeast is engineered to produce at least one product selected from the group consisting of organic acids, amino acids, vitamins, polyols, solvents, biofuels, therapeutics, vaccines, proteins, and peptides.

15. The method of claim 1, wherein the recombinant organism is a yeast, and wherein the yeast is engineered to produce organic acids.

16. The method of claim 1, wherein the recombinant organism is a yeast, and wherein the yeast is engineered to produce lactic acid.

17. The method of claim 1, wherein the recombinant organism is a bacterium and wherein the bacterium is engineered to produce at least one product selected from the group consisting of organic acids, amino acids, vitamins, polyols, solvents, biofuels, therapeutics, vaccines, proteins, and peptides.

18. The method of claim 1, wherein the recombinant organism is a bacterium and wherein the bacterium is engineered to produce organic acids.

19. The method of claim 5, wherein the recombinant organism is a bacterium and wherein the bacterium is engineered to produce lactic acid.

20. The method of claim 1, wherein the recombinant organism is a filamentous fungus and wherein the filamentous fungus is engineered to produce at least one product selected from the group consisting of citric acid, lactic acid, and enzymes.

21. A method of increasing stress tolerance in a recombinant organism that is engineered for industrial production of at least one product, comprising culturing the recombinant organism in a medium containing an effective amount of ascorbic acid.

22. The method of claim 21, wherein the effective amount of L-ascorbic acid is 0.005 to 2.0 grams/liter.

23. The method of claim 21 wherein the effective amount of L-ascorbic acid is 0.015 to 0.1 gram/liter.

24. The method of claim 21, wherein the recombinant organism is engineered for the industrial production of lactic acid.

25. The method of claim 21, wherein the recombinant organism is a bacterium, a yeast, a filamentous fungus, or an animal cell.

26. A method of increasing stress tolerance in an organism that produces lactic acid comprising culturing the organism in a medium containing 0.005 to 2.0 grams/liter of ascorbic acid.


SEQUENCE LISTING (15 sequences)

SEQ ID NO: 1 - protein, 377 aa, Arabidopsis thaliana
MGTTNGTDYGAYTYKELEREQYWPSENLKISITGAGGFIASHIARRLKHEGHYVIASDWKKNEHMTEDMFCDEFHLVDLRVMENCLKVTEGVDHVFNLAADMGGMGFIQSNHSVIMYNNTMISFNMIEAARINGIKRFFYASSACIYPEFKQLETTNVSLKESDAWPAEPQDAYGLEKLATEELCKHYNKDFGIECRIGRFHNIYGPFGTWKGGREKAPAAFCRKAQTSTDRFEMWGDGLQTRSFTFIDECVEGVLRLTKSDFREPVNIGSDEMVSMNEMAEMVLSFEEKKLPIHHIPGPEGVRGRNSDNNLIKEKLGWAPNMRLKEGLRITYFWIKEQIEKEKAKGSDVSLYGSSKVVGTQAPVQLGSLRAADGKE

SEQ ID NO: 2 - protein, 271 aa, Arabidopsis thaliana
MADNDSLDQFLAAAIDAAKKAGQIIRKGFYETKHVEHKGQVDLVTETDKGCEELVFNHLKQLFPNHKFIGEETTAAFGVTELTDEPTWIVDPLDGTTNFVHGFPFVCVSIGLTIGKVPVVGVVYNPIMEELFTGVQGKGAFLNGKRIKVSAQSELLTALLVTEAGTKRDKATLDDTTNRINSLLTKVRSLRMSGSCALDLCGVACGRVDIFYELGFGGPWDIAAGIVIVKEAGGLIFDPSGKDLDITSQRIAASNASLKELFAEALRLTGA

SEQ ID NO: 3 - DNA, 1134 bp, Arabidopsis thaliana
atgggaactaccaatggaacagactatggagcatacacatacaaggagctagaaagagagcaatattggccatctgagaatctcaagatatcaataacaggagctggaggtttcattgcatctcacattgctcgtcgtttgaagcacgaaggtcattacgtgattgcttctgactggaaaaagaatgaacacatgactgaagacatgttctgtgatgagttccatcttgttgatcttagggttatggagaattgtctcaaagttactgaaggagttgatcatgtttttaacttagctgctgatatgggtggtatgggttttatccagagtaatcactctgtgattatgtataataatactatgattagtttcaatatgattgaggctgctaggatcaatgggattaagaggttcttttatgcttcgagtgcttgtatctatccagagtttaagcagttggagactactaatgtgagcttgaaggagtcagatgcttggcctgcagagcctcaagatgcttatggtttggagaagcttgctacggaggagttgtgtaagcattacaacaaagattttggtattgagtgtcgaattggaaggttccataacatttatggtccttttggaacatggaaaggtggaagggagaaggctccagctgctttctgtaggaaggctcagacttccactgataggtttgagatgtggggagatgggcttcagacccgttcttttacctttatcgatgagtgtgttgaaggtgtactcaggttgacaaaatcagatttccgtgagccggtgaacatcggaagcgatgagatggtgagcatgaatgagatggctgagatggttctcagctttgaggaaaagaagcttccaattcaccacattcctggcccggaaggtgttcgtggtcgtaactcagacaacaatctgatcaaagaaaagcttggttgggctcctaatatgagattgaaggaggggcttagaataacctacttctggataaaggaacagatcgagaaagagaaagcaaagggaagcgatgtgtcgctttacgggtcatcaaaggtggttggaactcaagcaccggttcagctaggctcactccgcgcggctgatggaaaagagtga

SEQ ID NO: 4 - DNA, 1028 bp, Arabidopsis thaliana
ctaggctcgagaagcttgtcgacgaattcagatatccatggcggacaatgattctctagatcagtttttggctgccgccattgatgccgctaaaaaagctggacagatcattcgtaaagggttttacgagactaaacatgttgaacacaaaggccaggtggatttggtgacagagactgataaaggatgtgaagaacttgtgtttaatcatctcaagcagctctttcccaatcacaagttcattggagaagaaactacagctgcatttggtgtgacagaactaactgacgaaccaacttggattgttgatcctcttgatggaacaaccaatttcgttcacgggttccctttcgtgtgtgtttccattggacttacgattggaaaagtccctgttgttggagttgtttataatcctattatggaagagctattcaccggtgtccaagggaaaggagcattcttgaatggaaagcgaatcaaagtgtcagctcaaagcgaacttttaaccgctttgctcgtgacagaggcgggtactaaacgagataaagctacattagacgatacaaccaacagaatcaacagtttgctaaccaaggtcaggtcccttaggatgagtggttcgtgtgcactggacctctgtggcgttgcgtgtggaagggttgatatcttctacgagctcggtttcggtggtccatgggacattgcagcaggaattgtgatcgtgaaagaagctggtggactcatctttgatccatccggtaaagatttggacataacatcgcagaggatcgcggcttcaaacgcttctctcaaggagttattcgctgaggcgttgcggcttacaggggcatgattatcacgaattctggatccgatacgtaacgcgtctgcagcatgcgtggtaccgagcttttccctatagtgagtcgtattagagcttggcgtaatcatggtcatagctgtttcctgtgtgaattgttatccgctcacatttcacacaacatacgagccggaagcataaagtgtaa

SEQ ID NO: 5 - DNA, 1581 bp, Saccharomyces cerevisiae
atgtctactatcccatttagaaagaactatgtgttcaaaaactgggccggaatttattctgcaaaaccagaacgttacttccaaccaagttcaattgatgaggttgtcgagttagtaaagagtgccaggctagctgaaaaaagcttagttactgttggttcgggccattctcctagtaacatgtgcgttactgatgaatggcttgttaacttagacagattggacaaagtacaaaagtttgttgaatatcctgagttacattatgccgatgtcacagttgatgccggtatgaggctttaccaattgaatgaatttttgggtgcgaaaggttactctatccaaaatttaggctctatctcagaacaaagtgttgctggcataatctctactggtagtcatggttcctcaccttatcacggtttgatttcttctcaatacgtaaacttgactattgttaatggtaagggcgaattgaagttcttggatgccgaaaacgatccagaagtctttaaagctgctttactttcagttggaaaaattggtatcattgtctctgctactatcagggttgttcccggcttcaatattaaatccactcaagaagtgattacttttgaaaaccttttgaagcaatgggataccctatggacttcatctgaatttatcagagtttggtggtacccttatactagaaaatgtgttctatggaggggtaacaaaactacagatgcccaaaatggtccagccaagtcatggtggggtaccaagctgggtagatttttctacgaaactctattatggatctctaccaaaatctatgcgccattaaccccatttgtggaaaagttcgttttcaacaggcaatatgggaaattggagaagagctctactggtgatgttaatgttaccgattctatcagcggatttaatatggactgtttgttttcacaatttgttgatgaatgggggtgccctatggataatggtttggaagtcttacgttcattggatcattctattgcgcaggctgccataaacaaagaattttatgtccacgtgcctatggaagtccgttgctcaaatactacattaccttctgaacccttggatactagcaagagaacaaacaccagtcccggtcccgtttatggcaatgtgtgccgcccattcctggataacacaccatcccattgcagatttgctccgttggaaaatgttaccaacagtcagttgacgttgtacataaatgctaccatttataggccgtttggctgtaatactccaattcataaatggtttaccctttttgaaaatactatgatggtagcgggaggtaagccacattgggccaagaacttcctaggctcaaccactctagctgctggaccagtgaaaaaggatactgattacgatgactttgaaatgagggggatggcattgaaggttgaagaatggtatggcgaggatttgaaaaagttccggaaaataagaaaggagcaagatcccgataatgtattcttggcaaacaaacagtgggctatcataaatggtattatagatcctagtgagttgtccgactag

SEQ ID NO: 6 - DNA, 2138 bp, Saccharomyces cerevisiae
cccatgtctactatcccatttagaaagaactatgtgttcaaaaactgggccggaatttattctgcaaaaccagaacgttacttccaaccaagttcaattgatgaggttgtcgagttagtaaagagtgccaggctagctgaaaaaagcttagttactgttggttcgggccattctcctagtaacatgtgcgttactgatgaatggcttgttaacttagacagattggacaaagtacaaaagtttgttgaatatcctgagttacattatgccgatgtcacagttgatgccggtatgaggctttaccaattgaatgaatttttgggtgcgaaaggttactctatccaaaatttaggctctatctcagaacaaagtgttgctggcataatctctactggtagtcatggttcctcaccttatcacggtttgatttcttctcaatacgtaaacttgactattgttaatggtaagggcgaattgaagttcttggatgccgaaaacgatccagaagtctttaaagctgctttactttcagttggaaaaatcggtatcattgtctctgctactatcagggttgttcccggcttcaatattaaatccactcaagaagtgattacttttgaaaaccttttgaagcaatgggataccctatggacttcatctgaatttatcagagtttggtggtacccttatactagaaaatgtgttctatggaggggtaacaaaactacagatgcccaaaatggtccagccaagtcatggtggggtaccaagctgggtagatttttctacgaaactctattatggatctctaccaaaatctatgcgccattaaccccatttgtggaaaagttcgttttcaacaggcaatacgggaaattggagaagagctctactggtgatgttaatgttaccgattctatcagcggatttaatatggactgtttgttttcacaatttgttgatgaatgggggtgccctatggataatggtttggaagtcttacgttcattggatcattctattgcgcaggctgccataaacaaagaattttatgtccacgtgcctatggaagtccgttgctcaaatactacattaccttctgaacccttggatactagcaagagaacaaacaccagtcccggtcccgtttatggcaatgtgtgccgcccattcctggataacacaccatcccattgcagatttgctccgttggaaaatgttaccaacagtcagttgacgttgtacataaatcctaccatttataggccgtttggctgtaatactccaattcataaatggtttaccctttttgaaaatactatgatggtagcgggaggtaagccacattgggccaagaacttcctaggctcaaccactctagctgctggaccagtgaaaaaggatactgattacgatgactttgaaatgagggggatggcattgaaggttgaagaatggtatggcgaggatttgaaaaagttccggaaaataagaaaggagcaagatcccgataatgtattcttggcaaacaaacagtgggctatcataaatggtattatagatcctagtgagttgtccgactagtctctttttgtctcaataatctctatattttactaaaaaagaatatatatatatatatttatatatagcagtgtgatgactgttcatgtacattctaataactattcctagctgcctatcaaagacttttttttgaattagagctttttagtaatcatgggacccttttttcttttcattatccttactatagtttttttttggaaaagccgaacgcggtaatgattggtcgtataagcaaaaacgaaacatcggcatggcataacgtagatcctatctacagggaagtttttagaaatcagatagaaatgtattttgagtgctgtatatattgcagtactttttttctctctaggatttaagtatgtttagtattaactcatatcacattttttctttgtaaaaagcaaccattcgcaacaatgtcgatagtagagacatgcatatcgtttgtttcgacaaatccgttttatccattttgtactggattgcttctgaattgtgtggttacaccgctttacttttggaaaacgcaaaatggtagaatcgtggtc

SEQ ID NO: 7 - DNA, 960 bp, Arabidopsis thaliana
atgacgaaaatagagcttcgagctttggggaacacagggcttaaggttagcgccgttggttttggtgcctctccgctcggaagtgtcttcggtccagtcgccgaagatgatgccgtcgccaccgtgcgcgaggctttccgtctcggtatcaacttcttcgacacctccccgtattatggaggaacactgtctgagaaaatgcttggtaagggactaaaggctttgcaagtccctagaagtgactacattgtggctactaagtgtggtagatataaagaaggttttgatttcagtgctgagagagtaagaaagagtattgacgagagcttggagaggcttcagcttgattatgttgacatacttcattgccatgacattgagttcgggtctcttgatcagattgtgagtgaaacaattcctgctcttcagaaactgaaacaagaggggaagacccggttcattggtatcactggtcttccgttagatattttcacttatgttcttgatcgagtgcctccagggactgtcgatgtgatattgtcatactgtcattacggcgttaatgattcgacgttgctggatttactaccttacttgaagagcaaaggtgtgggtgtgataagtgcttctccattagcaatgggcctccttacagaacaaggtcctcctgaatggcaccctgcttcccctgagctcaagtctgcaagcaaagccgcagttgctcactgcaaatcaaagggcaagaagatcacaaagttagctctgcaatacagtttagcaaacaaggagatttcgtcggtgttggttgggatgagctctgtctcacaggtagaagaaaatgttgcagcagttacagagcttgaaagtctggggatggatcaagaaactctgtctgaggttgaagctattctcgagcctgtaaagaatctgacatggccaagtggaatccatcagaactaa

SEQ ID NO: 8 - DNA, 22 bp, Artificial Sequence
  Forward PCR Primer for D-arabinono-1,4-lactone oxidase from S. cerevisiae
tttcaccatatgtctactatcc

SEQ ID NO: 9 - DNA, 22 bp, Artificial Sequence
  Reverse PCR Primer for D-arabinono-1,4-lactone oxidase from S. cerevisiae
aaggatcctagtcggacaactc

SEQ ID NO: 10 - DNA, 23 bp, Artificial Sequence
  Forward PCR Primer for L-galactose dehydrogenase from A. thaliana
atgacgaaaatagagcttcgagc

SEQ ID NO: 11 - DNA, 24 bp, Artificial Sequence
  Reverse PCR Primer for L-galactose dehydrogenase from  A. thaliana
ttagttctgatggattccacttgg

SEQ ID NO: 12 - DNA, 26 bp, Artificial sequence
  Forward PCR Primer for D-mannose-L-galactose epimerase from Arabidopsis thaliana
gcgccatgggaactaccaatggaaca

SEQ ID NO: 13 - DNA, 25 bp, Artificial sequence
  Reverse PCR Primer for D-mannose-L-galactose epimerase from  A. thaliana
gcgctcgagtcactcttttccatca

SEQ ID NO: 14 - DNA, 23 bp, Artificial sequence
  Forward PCR Primer for myoinositol phosphatase from A. thaliana
atccatggcggacaatgattctc

SEQ ID NO: 15 - DNA, 21 bp, Artificial sequence
  Reverse PCR Primer for myoinositol phosphatase from  A. thaliana
aatcatgcccctgtaagccgc